(19) United States
(12) Patent Application Publication
(10) Pub. No.: US 2012/0270241 A1
(43) Pub. Date: Oct. 25, 2012

(54) SYSTEMS AND METHODS FOR EVOLVING ENZYMES WITH DESIRED ACTIVITIES

(76) Inventor: Philip N. Bryan, North Potomac, MD (US)

(21) Appl. No.: 13/497,753

(22) PCT Filed: Sep. 23, 2010
(86) PCT No.: PCT/US10/49992

§ 371 Date: Mar. 22, 2012

Related U.S. Application Data
(60) Provisional application No. 61/244,917, filed on Sep. 23, 2009.

(51) Int. Cl. C12N 9/96; C12N 9/50; G01N 21/64

(52) U.S. Cl. 435/7.72; 435/188; 435/219

(57) ABSTRACT

The present invention provides a new method for engineering or evolving enzymes to have desirable characteristics. Among the desirable characteristics is the ability to control catalytic activity through the use of a trigger molecule that rescues a catalytic site defect introduced during the engineering process. The method includes co-evolving enzyme and substrate to retain or improve substrate binding activity in the absence of catalytic activity.

### CROSS-REFERENCE TO RELATED APPLICATIONS

This application relies on and claims the benefit of the filing date of U.S. provisional patent application No. 61/244,917, filed 23 Sep. 2009, the entire disclosure of which is hereby incorporated herein by reference.

### STATEMENT OF GOVERNMENT INTEREST

This invention was made partially with U.S. Government support from the United States National Institutes of Health under grant/contract number NIH R44GM076786. The U.S. Government has certain rights in the invention.

### BACKGROUND OF THE INVENTION

1. Field of the Invention

The present invention relates to the field of biotechnology. More specifically, the invention relates to methods of engineering enzymes having catalytic activities that are controllable by small molecule effectors or triggers, engineered enzymes made by those methods, and methods of using the engineered enzymes.

2. Description of Related Art

Advances in biotechnology and protein biochemistry over the last two decades have provided researchers powerful tools to study enzyme expression and activity. Detailed knowledge is now available on the molecular bases for cellular production of enzymes of all types and activities, and on the molecular mechanisms of the catalytic activities of enzymes. Various enzymes having unique or beneficial properties have been discovered, isolated, purified, and studied. Among the many techniques widely used to study enzymes is the technique of mutagenesis, which can be used to dissect and analyze enzymes at the amino acid level to determine the functional and physical characteristics of enzymes.

Mutagenesis, performed either randomly or in a site-specific manner, is widely used to identify amino acid residues and combinations of residues that are important for enzymatic function. Due to the power and control afforded by molecular biology and protein biochemistry techniques, mutations can be introduced into enzymes, the mutations mapped precisely, and the effects of the mutations on enzyme structure and function determined. Typically, mutations affecting enzyme function are focused on the active site(s) of enzymes, and the effect of the mutations on substrate binding and catalysis detected.

Early mutagenesis studies focused on identifying particular residues that are involved in enzymatic activity. Recently, researchers have used mutagenesis to mutate enzymes in order to alter catalytic function, for example by improving substrate binding, by improving substrate specificity, or by improving catalytic activity. These enzyme engineering schemes have been loosely referred to as “in vitro evolution” of enzymes. Various “evolved” engineered enzymes are known in the art, and many have commercial value.

While the technology for engineering enzymes with beneficial attributes not possessed by the wild-type enzymes from which they are derived is robust, widely-practiced, and predictable, there still exists a need in the art for improved methods for engineering or “evolving” enzymes to obtain enzymes with desired characteristics. The present invention provides a new method for engineering enzymes having desired characteristics and additionally having catalytic activity that can be exquisitely controlled.

### SUMMARY OF THE INVENTION

The present invention provides methods of engineering enzymes. The methods are applicable to all enzymes having a detectable catalytic activity and having a known amino acid sequence, for example by way of a nucleic acid sequence encoding the enzyme. In general, the methods of engineering enzymes include mutating one or more residues that are involved in the catalytic function of the enzyme, such as at or near the catalytic site of the enzyme, to substantially reduce or eliminate catalytic function. The mutation(s) are created such that binding of a substrate of interest is not significantly decreased, and is preferably improved, while catalytic function is reduced or eliminated. As used herein, enzymes having “substantial” activity for a particular function are those that have at least about 70% of wild-type activity, preferably at least about 80%, more preferably at least about 90%, and most preferably at least about 99% of wild-type activity, as measured using an art-recognized assay for the particular function of interest. In some embodiments, the enzymes have 100% or greater than 100% of wild-type catalytic activity. In other embodiments, the catalytic activity is improved for a substrate that has a different structure than the “natural” substrate for the enzyme. While not so limited, the activity can be up to or exceeding 200%, 300%, 500%, 1000%, or more of wild-type activity. For example, activity can be 10-fold greater than wild-type activity, 20-fold greater, 50-fold greater, 100-fold greater, 500-fold greater, or 1000-fold greater. Likewise, an activity that is “substantially reduced” is one that shows a reduction in activity of at least about 30% of wild-type activity, preferably at least about 50%, more preferably at least about 75%, and most preferably at least about 90% of wild-type activity, as measured using an art-recognized assay for the particular function of interest. As used herein, the terms “substantially” and “significantly” are used synonymously with respect to activity. Further, as used herein, the term “essentially” when used with respect to activity indicates a level of from about 98% to about 100% of the activity to which it is compared. The term “essentially” is used to capture the concept of minor, insignificant changes in activity and the concept that experimental assays inherently have a level of error associated with them. Of course, any particular level of activity within these ranges is contemplated by the invention, and those of skill in the art will recognize this concept without the need for a specific disclosure of every particular value encompassed by these ranges. The mutations that are created are ones that can be complemented or “rescued” by externally provided substances, such as small molecules. According to the invention, these externally provided substances are referred to as “triggers” that, when provided, recapitulate the catalytic function of the mutated enzyme and thus generate a catalytically active enzyme. The methods allow for creation of engineered enzymes having substantial or even wild-type level substrate binding activity, but little or no intrinsic catalytic activity.

The unique properties engineered into enzymes can be used advantageously in methods of making the enzymes, in methods of isolating or purifying the enzymes, and in methods of using the enzymes. More specifically, the process of “evolving” enzymes according to the present invention typically is an iterative process in which one or more mutations are created in an enzyme, and the mutant enzymes assayed for one or more activities (e.g., catalysis in the presence of a “trigger”). The methods can also include purifying the mutant enzymes. Enzymes having desired characteristics are then subjected to one or more additional rounds of mutation and selection until a final engineered enzyme is evolved. The inability of the engineered enzymes to catalyze a selected reaction in the absence of an exogenously supplied trigger can be used in the method of making the enzymes by allowing selection of only those enzymes having a catalytic activity or level of catalytic activity that is regulated by the chosen trigger, and in selection of only those enzymes having a desired level of specificity for a given substrate. As detailed below, a phage display system that allows for selection of engineered enzymes is employed as part of the method of making engineered enzymes.

The present invention also provides for multiple uses of the engineered enzymes. Because the engineered enzymes of the invention are highly specific and tightly regulated with respect to their catalytic activities and substrate specificities, they can be used in any number of settings that benefit from temporal control of enzyme activity. It is known in the art that enzymatic activity can be controlled by controlling the environment of the enzyme. For example, enzymatic activity can be inhibited by raising or lowering the salt concentration around the enzyme, by raising or lowering (typically lowering) the temperature of the enzyme, by chelating metals or other co-factors, etc. As such, enzymes can be inactivated and maintained in an inactive state, then reactivated at a chosen time. The present invention provides a new way to temporally control enzymatic activity. However, unlike many other methods known in the art, the present methods of use allow for binding of inactivated enzymes to selected substrates. This characteristic can be highly advantageous, for example in purification schemes, enzyme kinetics assays, crystal structure analyses, analyte detection assays, and in creation of therapeutic “restriction proteases”, which inactivate key proteins in pathogens. In essence, an evolved enzyme of the invention can be used in any process or composition that a non-evolved corresponding enzyme (e.g., a wild-type enzyme) can be used. For example, the evolved enzymes of the invention can be used in enzyme-catalyzed synthetic reactions for production of useful products. Additionally, the methods of evolving enzymes can be used to create enzymes having novel activities. For example, enzymes can be evolved to have altered specificities that allow for catalytic activity on additional or alternative substrates (e.g., conversion of an enzyme requiring a high energy coenzyme-A substrate to an enzyme that can utilize ATP).

The invention provides enzymes engineered using the methods disclosed herein. Because the method of engineering or evolving enzymes is applicable to all enzymes with a detectable activity, the enzymes encompassed by the present invention are not particularly limited. In exemplary embodiments discussed below, the enzymes are proteases having known substrate cleavage sites or engineered to have specific substrate cleavage sites. According to the invention, the engineered enzymes are tightly regulated with respect to catalytic activity, having little, essentially no, or no detectable catalytic activity for a defined substrate. The enzymes have defined mutations that affect catalytic activity while at the same time the enzymes have substantial (approaching or achieving or surpassing wild-type) substrate binding activity. Preferably, the engineered enzymes have high specificity, approaching, achieving, or exceeding wild-type specificity. The enzymes have a cognate binding partner that is competent for substrate binding, but defective for catalysis until rescued or recapitulated by an exogenously supplied trigger.

The engineered enzymes of the invention can be provided as isolated or purified substances, as part of compositions, or as part of kits. When provided as part of compositions, the compositions include the enzymes and at least one other substance. The other substance is not particularly limited, but is preferably one that is compatible with the stability and function of the enzyme in the composition. Compositions thus may comprise, for example, water or an aqueous solution, mixture, etc. Buffers, salts, organic solvents, and other substances known in the art as compatible with enzyme storage and activity can be included in the compositions as well. In exemplary embodiments, the compositions comprise some or all of the substances necessary for assaying an activity of the engineered enzyme. In embodiments, the compositions comprise the enzyme in combination with a substrate and/or a trigger. When provided as a part of a kit, preferably the kit also includes the trigger molecule for the enzyme. Due to the various divergent uses of the enzymes of the invention, kits according to the invention can include any number of different components. In general, a kit according to the invention contains one or more engineered enzymes and some or all of the supplies and reagents for use of the enzyme in a particular application. Kits generally contain one or more containers to contain the enzyme, reaction reagents, and/or trigger. Kits can also contain solid supports for binding of the enzyme or substrate, or other reagents for practicing a method of the invention.

### BRIEF DESCRIPTION OF THE DRAWINGS

The accompanying drawings, which are incorporated in and constitute a part of this specification, illustrate embodiments of the invention, and together with the written description, serve to explain and provide data supporting certain principles of the invention.

FIG. 1 depicts a cartoon representation of the prodomain-SBT189 (subtilisin) interface, and its use in a method of engineering a triggered subtilisin according to the invention.

FIG. 2 shows a protein gel indicating the successful processing of the substrate “G_{B}-LFRAL-SA GFP” by subtilisin mutant SBT189.

FIG. 3 shows a plot of relative fluorescence over time to indicate activity of an engineered enzyme of the invention for its substrate.

FIG. 4 shows a representation of the crystal structure of a mutant subtilisin according to the invention.

FIG. 5 shows a representation of the release step in subtilisin phage display in which released phage in complex with “G_{A}-P_{COGNATE}” are bound to HSA-Sepharose.

FIG. 6 shows a representation of the amino acids comprising the S1 and S4 sub-sites of subtilisin.

FIG. 7 shows a representation of an anion site library in which substrate occupying the P4 to P2′ sub-site is shown. The bound anion is depicted as spheres. Active site residues are 32, 64, and 221. Sites of random mutagenesis are indicated with arrows.

FIG. 8, Panel A shows a plot of the kinetics of binding and cleavage of “G_{A}-P_{LFRAL-S}-G_{B}” by RSUB1(AF350), while Panels B and C show plots of cleavage kinetics for pre-formed “G_{A}-P_{LFRAL-S}-G_{B}”-RSUB1(AF350) complex monitored by fluorescence.

FIG. 9 depicts an activation cascade according to one embodiment of the invention. Depicted is a nitrite-triggered protease specific for the cognate amino acid sequence LFRAL-S (SEQ ID NO:1). The cognate sequence is engineered into the loop of a prodomain which specifically inhibits a second protease with a different cognate specificity. A FRET peptide with the second cognate sequence becomes fluorescent when cleaved by protease 2. If the second protease is triggered by a second anion, the signal will be generated only in the presence of both anions.

FIG. 10 depicts a line graph showing increase in fluorescence as a result of generation of active proteases through a proteolytic cascade reaction.

FIG. 11 depicts a reciprocal cascade scheme in which production of active protease is through a mechanism in which activated protease can not only generate a detectable signal via direct action on the detection label, but can also generate additional activated proteases via direct action on other proteases.

FIG. 12 depicts a serial activation scheme in which an active protease causes production of other active proteases, which then generate a detectable signal via action on another protease.

### DETAILED DESCRIPTION OF VARIOUS EMBODIMENTS OF THE INVENTION

Reference will now be made in detail to various exemplary embodiments of the invention, examples of which are illustrated in the accompanying drawings. The following detailed description focuses on exemplary embodiments of the invention and is provided to give the reader a better understanding of certain features of the invention. As such, it is not to be interpreted as a limitation on the scope of the invention. For example, while the following detailed description focuses on proteases as model enzymes, the invention is to be understood as applicable to all enzymes having a known catalytic function.

Before embodiments of the present invention are described in detail, it is to be understood that the terminology used herein is for the purpose of describing particular embodiments only, and is not intended to be limiting. Further, where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention

Unless defined otherwise, all technical and scientific terms used herein have th same meaning as commonly understood by one of ordinary skill in the art to which the term belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publications.

As used herein and in the appended claims, the singular forms “a”, “an”, and “the include plural referents unless the context clearly dictates otherwise. Thus, for example reference to “a mutant enzyme or an engineered enzyme” includes a plurality of such enzymes and reference to “the sample” include reference to one or more samples and equivalents thereof known to those skilled in the art, and so forth. Likewise, mention of “a mutation” indicates a single mutation or multiple mutations. The context of the disclosure will make evident whether a single or a plurality of items are envisioned.

It has long been recognized that the ability to engineer protease specificity would be a transformational technology. Consequently, this has been a goal of protein engineering efforts since the mid 1980s. While simple in concept, the mechanistic knowledge of proteases required to engineer their specificity is very complex and numerous factors cause the sequence specificity of currently known engineered proteases to fall short of that observed with natural processing proteases. A breakthrough described here is the understanding of how to link substrate binding energy and transition state stabilization by making proteolysis dependent on binding a small molecule co-factor that triggers proteolysis. This understanding provides the ability to engineer proteases that are both highly specific for defined sequence patterns in a substrate polypeptide and that are tightly regulated for catalytic activity with specific small molecules.

The ability to engineer high-specificity, tightly regulated proteases creates vast potential for building enzyme-based nanomachines. The protease occupies a central role in these nanomachines analogous to the role of a transistor in electronic devices. More specifically, a transistor uses a small change in current to produce a large change in voltage, current, or power, and allows the transistor to function as an amplifier or a switch in a circuit. In a similar manner, the regulable proteases of the present invention can function as either a switch or amplifier in a protein cascade, allowing complex output to be coupled to simple chemical signals. These protease-based devices can be understood in the context of the following simple scheme.

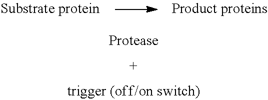

The substrate protein varies from application to application as does the triggering molecule. Examples of protease-base nanomachines to be described herein include three main areas of use: 1) protein purification and analysis; 2) small molecule detectors for medical diagnostics and bio-defense; and 3) therapeutic “restriction proteases” that inactivate key proteins in pathogens.

Subtilisin is a Bacillus subtilis serine protease whose natural function is to degrade proteins in the extracellular environment in order to provide amino acids to the soil-inhabiting bacteria. The enzyme is also an important industrial enzyme as well as a model for understanding enzymatic rate enhancements. For these reasons, together with the timely cloning of the gene and early availability of atomic resolution structures, subtilisin became an early model system for protein engineering studies. Although the Bacillus subtilis serine protease has been a popular model for protein engineering, engineering high specificity has proven problematic.

Previous studies with subtilisin have shown that mutating a catalytic amino acid invariably will drastically reduce catalytic activity. Studies with other enzymes have also shown that catalytic activity sometimes can be partially recovered in these mutants by adding a small molecule that mimics the chemical properties of the mutated catalytic amino acid. The inventor put these two observations together to create a subtilisin with a proto-binding site for fluoride. This mutant has useful properties and is described in co-pending U.S. patent application publication number 2006/0134740, which is incorporated herein by reference in its entirety.

Like the prior technology, the current invention also begins with a mutated catalytic amino acid, but the current invention further provides for reconfiguration of the active site to generate additional desired properties. For example, as compared to the prior work of the inventor, the present invention provides engineered enzymes with fully competent substrate binding regions, which have been evolved with a given substrate to ensure acceptable binding of that substrate without additional modifications to the substrate to support substrate binding to the active site. The present invention provides the first disclosure of engineered enzymes having mutated active sites that can be chemically rescued while at the same time retaining essentially wild-type levels of substrate specificity. In certain embodiments, the substrate specificity is for the “natural” or “normal” substrate of the enzyme, while in other embodiments, the specificity is for an alternative substrate. In embodiments involving alternative substrates, catalytic activity of the engineered/mutant enzyme is essentially the same as for the “natural” substrate and specificity for the alternative substrate is essentially the same as for the “natural” substrate. In some embodiments, catalytic activity and/or specificity of the engineered enzyme for the alternative substrate is higher than for the “natural” substrate.

The present disclosure teaches how to produce high-specificity, tightly regulated enzymes. The first two steps in this process have been disclosed in the art. (See, for example, Craik et al., 1987; Ruan et al., 2004; Toney and Kirsch, 1989.) The first step is to mutate a critical amino acid in the active site of the target enzyme. Mutation of a critical amino acid reduces or abolishes catalytic activity of the mutant enzyme. In conjunction with the mutagenesis step, a second step is performed to identify a co-factor that increases catalytic activity when added to the mutant enzyme and a cognate substrate. A suitable co-factor is a molecule that mimics the chemical properties of the mutated critical amino acid. That is, the co-factor provides chemical and physical properties that replace the chemical and physical properties of the catalytic site that were lost due to changing the critical residue to a different residue. The mutant enzyme is referred to herein as a “triggered enzyme” and the co-factor is referred to herein as the “trigger”. The present invention improves on this basic method by showing how co-factor dependence can create high specificity and by teaching how to co-evolve the enzyme, the trigger, and the substrate together to generate enzymes that are robust, highly specific, and tightly regulated. This concept is illustrated below in the Examples using the serine protease subtilisin.

The present invention provides numerous benefits to efforts toward enzyme engineering. Among the benefits, mention may be made of: use in protein purification and analysis; creation of small molecule detectors for medical diagnostics and bio-defense; and creation of therapeutic “restriction proteases”.

In a first general aspect, the present invention provides methods of engineering or evolving enzymes. The method includes mutating one or more residues at or near the catalytic site of an enzyme to substantially reduce or eliminate catalytic function. Typically, one or more residues that are required for catalytic activity of the enzyme are mutated to abolish or substantially reduce catalytic activity for a pre-selected substrate. In some embodiments, one or more specific residues previously identified as required for catalytic activity are mutated. In exemplary embodiments, a single residue involved in the catalytic function of the enzyme is mutated. In embodiments, site-directed mutagenesis is used to alter a particular, pre-selected residue. In other embodiments, random or pseudo-random mutagenesis is performed to mutate one or more residues of the enzyme, and the catalytic activity of mutant enzymes is assayed to identify mutants lacking catalytic activity. Preferably, a single residue is mutated.

As with known enzyme engineering methods, the method of enzyme engineering according to the present invention includes a selection step in which mutants having desired characteristics (e.g., lack of catalytic function) are identified and purified away from other mutants or wild-type enzymes. However, the present invention employs a novel selection process (discussed below), which is a powerful process that significantly reduces the amount of work required to identify and isolate mutants of interest. As with other methods of enzyme engineering, the method of the invention can include analyzing selected mutants for their amino acid sequences, typically by way of sequencing or PCR/restriction analysis of the selected mutants. Such analysis is routine in the enzyme engineering art, and does not represent undue or excessive experimentation. Indeed, because the present invention provides a powerful selection step, the amount of analysis performed to identify mutants of interest is substantially reduced as compared to prior art methods.

The method of engineering enzymes according to the invention is typically an iterative method that involves at least two rounds of mutation, selection, and characterization. As such, in embodiments, the method includes isolating a mutant enzyme of interest and subjecting it to one or more rounds of mutation, selection, and isolation. The subsequent rounds of mutation, selection, and isolation can be performed to further mutate a particular residue identified as catalytically important. However, in preferred embodiments, the subsequent rounds are performed to alternatively or additionally mutate non-catalytic residues of the enzyme. In a typical engineering process, catalytic destruction is accompanied by mutation of other residues of the enzyme pro-domain to retain or improve substrate binding and/or specificity. This co-evolution departs from prior art attempts at enzyme evolution, which focus only on mutation of the catalytic site. In essence, the method for engineering an enzyme according to the present invention involves creating a mutation at a catalytically important residue to reduce or abolish catalytic activity for a pre-defined substrate, and creating one or more additional mutations to improve specificity of the engineered enzyme for the pre-defined substrate.

It has been discovered by the inventor that co-evolution of a catalytic mutant for both catalytic function and substrate specificity provides a powerful means for providing an engineered enzyme having the ability to be catalytically regulated by an external substance, while at the same time providing an enzyme with wild-type or better substrate specificity. Furthermore, because mutants generated by the process must be isolated and analyzed at each round of mutation, screening for two or more mutations in the same enzyme requires little, if any, additional work. Prior attempts at enzyme engineering have been able to develop mutant enzymes that are catalytically controllable by external molecules; however, those enzymes had lower than wild-type substrate binding activity, which detracts from their usefulness for commercial or research purposes. The present invention overcomes this drawback.

According to the method of engineering enzymes, one or more mutations in the enzyme prodomain are introduced into the mutant enzymes to maintain or improve substrate binding and/or substrate specificity. Typically, the mutation(s) are those that improve the substrate binding pocket to overcome the structural change in the substrate binding pocket caused by the mutation of the catalytic residue(s). More specifically, it is understood in the art that a substrate binding site provides a three-dimensional structure that accommodates a substrate such that it is positioned for catalysis. Disruption of a binding site residue is generally thought to alter the three-dimensional structure of the binding site such that substrate binding, substrate specificity, catalysis, or two or all three of these are reduced. The method according to the present invention includes making one or more amino acid changes in the enzyme prodomain that counteracts the destabilizing effect of catalytic site residue mutation. As such, the engineered enzyme is catalytically deficient or defective but retains full substrate binding activity and specificity. Of course, the practitioner may elect to retain both substrate binding and substrate specificity, or may elect to retain only one of these characteristics. According to the invention, the method is practiced preferably to retain at least the substrate binding activity of the enzyme. Those of skill in the art will immediately recognize the advantages in some circumstances for a catalytically controllable enzyme having a lower than wild-type substrate specificity. For example, in some situations it can be desirable to create an engineered enzyme that has general specificity for two or more substrates of the same general class (e.g., binding of both RNA and DNA, binding of both single-stranded nucleic acid and double-stranded nucleic acid, etc.) rather than retaining or improving the specificity of the enzyme for its wild-type substrate. Those of skill in the art will also recognize the usefulness of creating mutant enzymes having altered specificity, in which the specificity of the enzyme for its “natural” substrate is reduced by the specificity for an alternative substrate is increased.

According to the method of the invention, an enzyme is engineered to have a catalytic function that is reduced or, preferably, abolished. The catalytic function is rescued by a second substance (a trigger). While any number of triggers can be used according to the invention, non-limiting examples include ions, such as fluoride, and small molecules, such as nitrite, formate, acetate, glycolate, lactate, pyruvate, and methylphosphonate. Other classes of molecules that can rescue function include nucleophiles (e.g., hydroxylamine), general bases (e.g., imidazole), and metals. In general it can be expected that the deletion of an acidic amino acid such as aspartic acid or glutamic acid can be compensated by small weak acids, such as fluoride, nitrite, lactate, etc. It can also reasonably be expected that mutating an amino acid which serves as a nucleophile in an enzymatic reaction (such as serine, cysteine or threonine) can be compensated by an exogeneous nucleophile such as hydroxylamine (and many other examples). Likewise a general base such as histidine can likely be compensated by a general base such as imidazole. Appropriate candidates for a triggering molecule can be anticipated base on well-established principles of chemistry. The degree to which any triggering molecule restores activity will also depend on the ability of the enzyme structure to accommodate the trigger, as well as the mutations introduced into the enzyme that create affinity for that trigger. The mutations needed to bind the triggering molecule in the correct way can be identified using the methods described here. However, because the present invention provides a powerful selection process, identifying appropriate mutation-trigger combinations can be performed easily without any prior trial-and-error experimentation. In general, the invention contemplates any trigger molecule that can function in conjunction with a mutant residue to provide the function of the wild-type catalytic residue. The trigger thus can be a small molecule that is positively charged that can substitute for the positive charge of a mutated lysine or arginine. Likewise, the trigger can be a small molecule that is negatively charged and can substitute for the negative charge of a mutated glutamic acid or aspartic acid. Additionally, a trigger containing a phenyl group can substitute for a mutated phenylalanine or tyrosine. Exemplary combinations of small molecules and corresponding mutant residues that recapitulate certain mutated residues are provided below in the Examples.

It is to be understood that the present invention relates to methods of co-evolving an enzyme and a substrate. More specifically, the invention provides a powerful method for engineering enzymes based on a known substrate, in which mutant enzymes are created and refined based on an ability to bind a given substrate and catalyze a reaction involving that substrate. Catalysis is regulated or controlled based on rescue of a catalytically defective enzyme using a trigger. However, in certain embodiments of the invention, the particular substrate is not the key factor in evolving the enzyme. Rather, in certain embodiments, the ability of an engineered enzyme to detect the presence of the trigger is the focus of the method. As such, in embodiments, the enzyme and the substrate can be co-evolved to develop a combination that is highly specific and highly sensitive to a pre-selected trigger. These embodiments generally relate to detection of small molecules that are indicative of a certain chemical or biological. For example, certain chemicals that can be used as poisons or in chemical warfare can be detected directly or indirectly by the presence in samples of small molecules that result from production or breakdown of the chemicals. Co-evolved enzyme/substrate combinations can be used to detect, with high sensitivity, these signature small molecules. Likewise, biological agents, such as pathogenic bacteria, produce or cause production of small molecules during infection. These small molecules can be detected using co-evolved enzyme/substrate combinations. Also, detection of natural metabolites found in cells and body fluids can be used to create a metabolic profile indicative of health or a specific disease state. A non-limiting example of such an assay for a chemical or biological involves the use of a labeled substrate that serves as a substrate for an engineered enzyme, in which the labeled substrate is bound to the enzyme in the absence of the chemical or biological. The enzyme could be bound to a solid support or the label could be quenched by its association with the enzyme and/or substrate. Upon exposure to the chemical or biological, the catalytic activity of the enzyme is restored and the label is cleaved from the substrate as a result proteolysis by the enzyme. The label is then detectable in solution.

The method of engineering enzymes includes a novel procedure for identifying mutants of interest. Prior art methods of enzyme engineering generally involve expression of a mutant form of an enzyme, binding of the enzyme to a solid matrix, then releasing the mutant enzyme for characterization and, optionally, further mutation. The prior art methods are time-consuming and labor intensive, in part due to the need to screen multiple mutants to identify those of interest. Moreover previous methods release mutant enzymes by disruption a binding interaction and not by directly selecting the ability to perform a chemical transformation (e.g., bond cleavage or formation). This difference is elaborated in more detail below. In contrast to the prior art methods, the present invention uses a selection process that involves a powerful catch and release phage display system to screen for mutants of interest.

Evolving enzymes by phage display is difficult because the technique selects for binding rather than catalysis. To try to circumvent this issue, transition-state analogues or suicide substrates are typically used in selection for enzymatic function. Because its selection is less direct, evolving enzymatic function has been much less successful than selecting for binding activity. The present invention addresses this shortcoming by using a catch and release phage display system that uses a combination of binding and catalysis to select for mutant enzymes. The ability to isolate substrate binding from substrate hydrolysis via a co-factor requirement (i.e., trigger), combined with the ability to display either the substrate or the engineered enzyme on the surface of a phage particle, presents an unprecedented opportunity to create novel enzymatic properties by directed evolution. The method of the present invention fundamentally differs from normal phage display methods, which amplify desired sequences only on the basis of selective binding. In the present catch and release system, binding of mutants is permissive and amplification of mutants with the desired activity is achieved by selective catalysis (e.g., hydrolysis of a fusion protein substrate) under a defined triggering condition. By further mutating the enzymes to improve substrate binding/specificity, the invention further improves prior art techniques by allowing selection based not only on catalytic activity, but on the level of specificity as well.

More specifically, the present invention provides for a phage display system that allows selection of enzymes based not only on the ability of the enzyme to bind a substrate, but also on the ability of the enzyme to catalyze a reaction. In particular, the present invention provides a phage display system that identifies an enzyme of interest based on its ability to bind a particular substrate. However, rather than simple release of the enzyme from the substrate as seen in other phage display systems, the present system utilizes the controlled or triggered catalytic activity to release the enzyme and substrate from each other.

Certain features of the catch and release phage display system of the invention will be explained now with reference to engineering of a protease. It is to be understood that, according to the invention as it relates to proteases, either the engineered enzyme or the substrate can be expressed using phage display technology, although the present discussion focuses on phage display of the enzyme. The initial process of phage display includes fusing a coding region of an enzyme to the coding region of a phage coat protein and producing recombinant phage in a suitable host. Phage thus express the engineered enzyme on their surface. Phage producing enzymes are captured through the interaction between the mutant enzyme on the phage surface with a substrate for the mutant enzyme, which is typically attached to a solid support. Non-binding phage are removed. In this step, the washing conditions can be adjusted to remove weakly binding mutant enzymes as well: the stringency of the wash can be adjusted as desired. This feature is particularly useful in rounds of selection where mutations have been created to improve enzyme specificity or binding for the substrate. In the next step, the catalytic activity of the mutant enzyme is rescued by exposure of the enzyme-substrate complex to a trigger. The trigger recapitulates the mutated catalytic site and causes the enzyme to cleave the substrate, releasing the phage from the solid support. The phage are then recovered and isolated. Isolated phage can be analyzed to determine the mutations present in the mutant enzymes. Phage of interest are selected and one or more further rounds of mutagenesis, capture, and, optionally analysis, are performed.

Co-evolving enzymes with substrates allows for creation of engineered enzymes having high specificity for a target substrate and little or no catalytic activity on that substrate. The engineered enzymes find use in multiple applications. For example, the engineered enzymes can be used to purify any number of proteins. In embodiments where engineered enzymes are used in purification schemes, the engineered enzyme are typically proteases, which are bound to a solid support. The co-evolved substrate peptide is fused to a protein of interest for purification. Binding of the protein of interest to the engineered enzyme occurs via the co-evolved peptide portion. Non-binding or poorly binding substances are washed from the solid support complex, then a trigger is supplied. The trigger activates the evolved enzyme, which cleaves the peptide substrate, releasing the protein of interest.

In other embodiments, the engineered enzymes can be used to detect a small molecule of interest, such as one indicative of a chemical or biological substance of interest. In these embodiments, a co-evolved enzyme/substrate combination can be created by binding of the enzyme to the substrate (one of which can be bound to a solid support) to create a complex. Exposure of the complex to a sample suspected of containing the substance of interest activates the catalytic activity of the enzyme, and causes cleavage of the substrate. Cleavage of the substrate can be monitored in any number of ways known in the art. For example, the substrate can be labeled and cleavage of the substrate can release the label from a solid support-bound enzyme/substrate, allowing for detection of the label in solution rather than as a support-bound entity. Alternatively, cleavage could release a portion of the substrate that was previously masking the signal of the label, allowing for detection. Numerous other detection methods for various enzymatic activities can be used. Where a protease is used, cleavage is indicative of the presence of the substance of interest in the sample. These embodiments are particularly useful in detecting small molecules that are derived from chemical weapons, poisons, and biological or biochemical molecules produced or caused to be produced by infectious agents. These embodiments thus have application in chemical warfare and bioterrorism protection.

In some embodiments, the co-evolved enzyme-substrate combination finds use in the creation of therapeutic restriction proteases. In these embodiments, proteases are engineered to have triggered protease activity for biologically-derived peptide substrates, which are indicative of a particular infectious agent. For example, proteases can be engineered with high specificity for peptide toxins (e.g., cholera toxin, diphtheria toxin, C. difficile toxin A or toxin B, etc.). The evolved enzymes can be used, among other things, to destroy the peptide substrates under controlled conditions.

In embodiments, the protease is a nanomachine used within a living organism to convert a specific pathogen protein into an inactive and benign form. The engineered restriction proteases are analogous to restriction endonucleases which were discovered by their ability to “restrict” invasion of bacteria by certain bacteriophages. Restriction endonucleases prevent infection by specifically cleaving foreign DNA. The restriction protease acts by selectively cleaving a pathogen protein involved in virulence. The ultimate goal is to create a new class of therapeutic molecules. In principle a specific restriction protease can be evolved to destroy a specific pathogen protein from any infectious agent. The molecule works like a traditional antibody in that it targets a specific epitope within the target protein. Unlike an antibody, which functions by stoichiometric binding, the restriction protease works catalytically and each protease molecule is capable of destroying thousands of target proteins. A restriction protease does not require high affinity for a target protein (like an antibody or a small molecule drug), but does need to be highly specific for the cognate sequence within the target protein.

Yet again, the engineered enzymes can be useful in proteomic analysis. A suite of site-specific proteases that cut with high specificity but different frequency would be powerful tools for proteomic analysis. The basic idea is to cut a sub-population of proteins that contain a specific sequence motif and then to resolve the population of cleaved proteins from the uncleaved. This produces a sequence-filtered slice of a proteome. The identity of this subset of proteins can be determined from searching protein databases for the cognate motif. In this application of the invention, the input is a biological extract (e.g., proteome). The output is cleaved proteins in that proteome which contain the cognate sequence motif. The regulator can be any of the small trigger molecules discussed herein and the like.

Two basic characteristics will determine the effectiveness of a protease for this type of proteomic analysis: 1) Frequency—how often the cognate motif occurs in a proteome; and 2) Specificity—the activity of the protease against the cognate motif relative to others. Frequency determines resolution. When every protein is cut, there is no resolution in the sequence dimension. A protease such as trypsin, while ideal for fingerprinting, has no resolving power because it cuts within virtually all proteins. The lower the frequency of cutting, the higher the resolving power of the protease. At the extreme, a protease may by engineered to cut only a single protein (e.g., a biomarker) in a given proteome allowing its detection without fractionation. The specificity of the protease determines the background it produces. The higher the specificity, the greater the ability of the protease to detect low abundance proteins in a complex mixture.

An additional requirement for a proteomics protease is stability in denaturing conditions. Denaturation removes the structural elements in target proteins and allows the protease to act based on primary sequence alone. The present invention has already established that proteases selected by catch and release techniques are thermostable and highly active in 0.1% SDS.

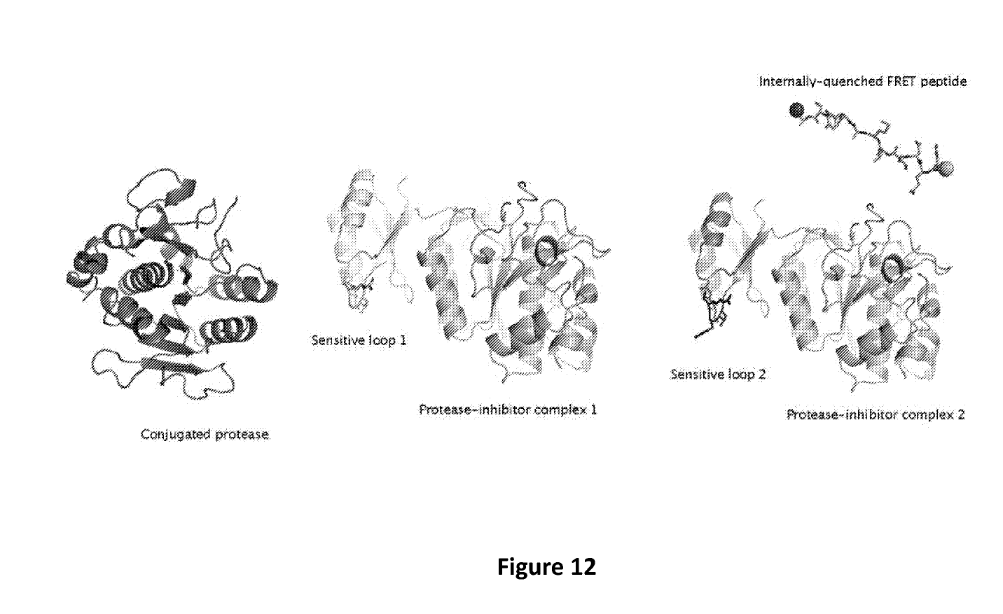

Certain embodiments of the invention involve use of one or more engineered proteases together in a detection scheme that enables one to detect small numbers of a molecule of interest through the use of an amplification reaction in which proteolysis by one protease activates multiple other proteases, all of which are capable of generating a signal. A powerful detection system can be built from four basic components: 1) a protease conjugated to a binding molecule, 2) an unconjugated protease, 3) an inhibitor protein that contains a proteolytic cleavage site, and 4) a protease substrate that generates a signal upon its cleavage. Versions of this system are depicted in FIGS. 19-12, discussed in detail below.

The present invention addresses unsolved problems in the art of enzyme engineering, and relies, at least in part, on the realization that co-factor binding and activation of enzymatic activity results in specificity that can be controlled or at least selected for. The conformation of a substrate in a ground state complex with an enzyme is similar but not identical to its conformation in the transition state. As a result, substrates that bind best in the ground state are not necessarily the fastest in the chemical transformations. Interactions of the substrate with the enzyme binding pocket must achieve an optimum balance between substrate binding and transition state stabilization. Further, enzymes generally impose very stringent geometric constraints on productive substrate interactions. Consequently, minor structural changes caused by mutation have large (and usually detrimental) effects on catalytic activity. By replacing an active site residue with a co-factor, the structural and mechanistic restraints on the way an enzyme can productively interact with a substrate are relaxed. The co-factor is free to adapt to the new active site with more freedom than an amino acid functional group (which is constrained by attachment to the main chain). When properly evolved or engineered, co-factor position can adjust to fit a new substrate, and substrate-enzyme interactions can be adjusted to a co-factor-dependent active site. This allows for the creation of altered specificities that would not have been possible in the context of a highly-constrained wild type active site.

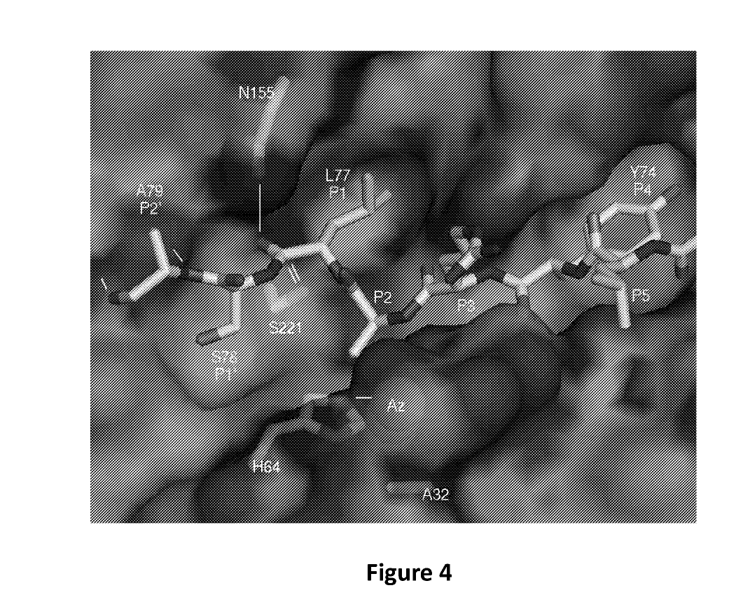

Prior attempts at protein engineering have met with limited success. Such attempts at protein engineering have not generally lead to highly functioning enzymes because enzyme catalysis is subtle and complex to understand, much less to engineer. This fact can be exemplified by analyzing the engineering of subtilisin. It is possible to engineer well-articulated binding pockets with apparent lock and key fit for amino acid sub-sites within a target substrate sequence (see FIG. 4, for example). The sequence specificity of subtilisin engineered in this way falls far short of that observed with natural processing proteases, however. The basic problem is that the desired cognate sequence may bind better than other sequences, but it is not turned-over much faster than non-cognate sequences. Consider a recent example (Knight, 2007), in which subtilisin was evolved to hydrolyze a substrate with phosphotyrosine at the P1 position. Native subtilisin hydrolyzes phosphotyrosine at P1 very poorly while the evolved enzyme hydrolyzes it very well. This is an impressive achievement. The problem is that activity against non-cognate P1 amino acids remains high in the engineered enzyme, which detracts from the engineered enzyme's usefulness.

A common assumption in enzyme engineering is that substrate binding is in rapid equilibrium and that the first chemical step (acylation for serine proteases) is rate limiting. These assumptions are often considered axiomatic for subtilisins, but in fact are not true for many substrate sequences. As substrate binding improves, these assumptions break down. To effectively engineer specificity one must balance the flux of species through the reaction pathway such that acylation is the rate limiting step and that substrate binding is kinetically uncoupled from acylation. The mechanistic basis for this fact is straightforward, although not generally considered by protein designers. The necessity of controlling relative affinities for substrates, transition states, intermediates, and products is addressed in detail in Ruan et al. (2008) for engineering specificity in subtilisin.

A second requirement for engineering serine protease specificity is to make the acylation rate strongly dependent on the desired cognate sequence. This is obviously true but difficult to engineer. The present invention provides a surprising solution to both problems by mutating an active site residue and selecting a cognate sequence that is best for the mutated active site. Obviously, mutating an active site residue radically decreases constitutive activity of an enzyme, but can allow for recovery of the lost activity through an exogenous small molecule that mimics the substituted amino acid (see, for example, Toney, 1989; Harpel, 1994; and Takahashi, 2006). In subtilisin, the inventor and his collaborators have previously mutated the catalytic D32 and rescued activity with specific small anions (e.g., azide or nitrite). While chemical rescue to investigate enzyme mechanisms is well known, engineering high functioning enzymes around an engineered co-factor dependence is novel. A common but erroneous assumption is that the resulting engineered enzymes will be slow. Depending on the anion and its concentration, wild type rates of acylation can be achieved, although this is not necessarily desirable for high specificity. The engineering problem is not in maintaining the maximum hydrolysis rate for a desired cognate sequence. The problem is discrimination among similar sequences. Employing an anion co-factor to trigger hydrolysis results in three benefits 1) the ability to maintain the protease in a virtual off-state in the absence of the anion; 2) the ability to appropriately tune the chemical steps relative to the binding steps (and thus control the flux of species through the reaction pathway by the anion concentration); and 3) the ability to optimize the effect of a substrate sequence on transition state stabilization rather than ground state stabilization (as described herein).

There are three basic challenges in selecting good proteases by directed evolution. First, one must go deep into sequence space. There are elegant methods for evolving enzymes in general (see, for example, Bloom, 2009) and proteases in particular (see, for example, Varadarajan, 2005) by introducing mutations with error prone PCR and reshuffling them with molecular breeding methods. There are also increasing sophisticated methods for screening these libraries for enzymatic function. These approaches works quite well for evolving stability (see, for example, Bryan, 1986; Pantoliano, 1989) and moderately well for improving catalytic activity for a desired substrate relative to the original wild type activity. They are largely disappointing, however, for evolving protease specificity (Pogson, 2009). The relevant question to ask is whether a desired property can be improved incrementally by the accretion of single mutational events (Bloom, 2009). To evolve high-specificity one needs to go deeper in sequence space than is possible with typical methods for mutagenesis and screening because many interdependent mutational events are required to achieve adequate solutions to the specificity puzzle.

The second basic challenge is that methods that maximize substrate binding affinity are not productive. The conformation of a peptide substrate in a ground state complex with the protease is similar but not identical to its conformation in the transition state. This is obviously true at the scissile bond itself, but these differences are propagated along the amino acid chain to the side chain sub-sites. As a result, the sequences that bind best in the ground state are not the fastest in the chemical transformations (see, for example, Hedstrom, 2002). In order to achieve efficient hydrolysis, the scissile bond of the substrate, the catalytic residues of the enzyme (H64, N155 and S221 for subtilisin), and the anion must be brought into precise register. Side chains of the substrate must control the position of the backbone through their interactions with the enzyme binding pockets to achieve the optimum balance between substrate binding and transition state stabilization. The screening method must be able to make this subtle discrimination. This creates a dilemma. In display methods such as phage display or ribosome display≧10 variants can be screened. This allows explorations deep in sequence space if the mutations are targeted to a well defined region such as a binding pocket. The problem is that normal phage display methods amplify desired sequences on the basis of binding alone. Because the present invention provides the ability to control peptide hydrolysis with an on-off switch, a method is now available in which selection is based on hydrolysis of a fusion protein in response to a trigger (e.g., an anion). Binding of the substrate is required but not sufficient for selection. The selection system acts as a sophisticated analogue computer which parses the sea of sequence space and finds enzymatic solutions that are extremely subtle and that are well beyond the state of the computational art.

The third basic challenge is to address the fact that the desired enzyme might be toxic to cells. Protease evolution presents unique problems because the desired phenotype can be toxic. This is well-documented and, in itself, an indication of the potential biological effects of a restriction protease. Negative selection is especially problematic during intermediate stages of evolution during which proteases have relaxed specificity. The present invention addresses this challenge through the use of triggering. Triggering allows protease activity to be off during the phage propagation phases of selection and turned on only during the in vitro phases of the process.

The present invention thus provides a unique and powerful method for engineering enzymes having desired activities on known substrates. In preferred embodiments, the methods comprise creating a mutation at a residue that participates in the catalytic function of the enzyme for a chosen substrate to reduce or abolish the catalytic activity of the enzyme for that substrate, wherein the catalytic activity of the mutant enzyme for that substrate can be restored by an exogenous trigger molecule; and creating another mutation in the enzyme, wherein the other mutation increases the catalytic activity and specificity of the mutant enzyme for a pre-selected substrate in the presence of the exogenous trigger molecule. Exemplary embodiments relate to proteases, such as the well-studied serine proteases, including, but not limited to subtilisin. In some embodiments of the method, the chosen substrate and the pre-selected substrate are different substrates, indicating that the method can be a method of engineering an enzyme for a particular substrate or a method of co-engineering an enzyme and a substrate. A powerful embodiment of the method includes a phage catch and release process as follows: expressing the mutant enzyme on the surface of a phage; binding the phage to the substrate, which is bound to a solid support; removing unbound phage; and exposing the enzyme-substrate complex to the trigger molecule to release the phage from the substrate. The method can further include recovering the phage that expresses the mutant enzyme and/or performing the phage catch and release process one or more additional times. Alternatively, each of the method steps can be performed one or more additional times.

The method of the present invention can also be considered as a method for identifying and isolating an engineered enzyme having the ability to bind a substrate of interest and catalyze a reaction involving that substrate, where the method includes the following steps: (a) creating a mutation at a residue that participates in the catalytic function of the enzyme for a chosen substrate to reduce or abolish the catalytic activity of the enzyme for that substrate, wherein the catalytic activity of the mutant enzyme for the chosen substrate can be restored by an exogenous trigger molecule; (b) creating another mutation in the mutant enzyme, wherein the other mutation increases the catalytic activity and specificity of the mutant enzyme for a pre-selected substrate; (c) expressing the mutant enzyme on the surface of a phage; (d) binding the phage to the pre-selected substrate, which is bound to a solid support; (e) exposing the enzyme-substrate complex to the trigger to release the phage from the pre-selected substrate; and (f) recovering the phage that expresses the mutant enzyme. The method can be practice in an embodiment where steps (b)-(f) are repeated one or more times using the sequence of the mutant enzyme obtained in step (f) of the previous cycle as the starting sequence for creating one or more other mutations, or where steps (c)-(f) are repeated one or more times.

The method of the present invention can also be considered as a method for engineering an enzyme for use in detection of a substance of interest, where the method includes the following steps: creating a mutation at a residue that participates in the catalytic function of the enzyme for a chosen substrate to reduce or abolish the catalytic activity of the enzyme for that substrate, wherein the catalytic activity of the mutant enzyme for that substrate can be restored by the substance of interest; and creating another mutation in the enzyme, wherein the other mutation increases the catalytic activity and specificity of the mutant enzyme for a pre-selected substrate in the presence of the substance of interest. In embodiments of the method, the chosen substrate and the pre-selected substrate are different substrates. In some embodiments, the method additionally includes expressing the mutant enzyme on the surface of a phage; binding the phage to the pre-selected substrate, which is bound to a solid support; exposing the enzyme-substrate complex to the trigger to release the phage from the pre-selected substrate; and recovering the phage that expresses the mutant enzyme.

In an embodiment of the invention, a method for detecting the presence of a substance of interest in a sample is provided. In essence, this embodiment uses an engineered enzyme, which is specific for a pre-defined substrate, to detect the presence of that substrate in a sample. In general, the method includes the following steps: forming a complex between the engineered enzyme and the substrate for the enzyme; exposing the complex to the sample, for example, by mixing the two together; and determining if the sample contains the substance of interest by detecting an increase in catalytic activity of the enzyme in the presence of the sample. In embodiments, the method is a method of detecting the presence in the sample of a molecule that is indicative of a chemical warfare agent, a poison, or a biological or biochemical product indicative of a harmful organism. For example, the method can be a method of detecting a biological or biochemical product that is a polypeptide toxin produced by a bacterium. Likewise, the method can be a method of detecting a charged molecule that is a breakdown product of a chemical warfare agent or poison.

Using the powerful engineering method of the invention, one may obtain an engineered (mutant) enzyme that is competent for substrate binding but defective for substrate catalysis in the absence of an exogenous trigger molecule, wherein the enzyme has the following characteristics: a mutation at a residue that is involved in the catalytic activity of the enzyme, which reduces or abolishes the catalytic activity of the enzyme for a chosen substrate, wherein the catalytic activity of the mutant enzyme can be restored by the exogenous trigger molecule; and another mutation in the mutant enzyme, wherein the other mutation increased the catalytic activity and specificity of the mutant enzyme for a pre-selected substrate in the presence of the trigger molecule. As should be evident from the description of the method of the invention, the chosen substrate and the pre-selected substrate can be different substrates. In exemplary embodiments, the engineered enzyme is a protease, such as a serine protease, including, but not limited to, subtilisin.

The engineered enzyme can be present as an isolated or purified substance, or can be part of a composition that also includes at least one other substance that is compatible with the catalytic activity of the engineered enzyme. In exemplary embodiments, the other substance is a trigger molecule that restores the catalytic activity of the engineered enzyme. Of course, the purified/isolated engineered enzyme and the composition can be provided as part of a kit, which preferably also includes the appropriate trigger molecule that restores the catalytic activity of the particular engineered enzyme of the kit.

The invention also provides for a protease-inhibitor protein complex having the following characteristics: the inhibitor protein contains a proteolytic cleavage site; cleavage of the inhibitor protein at the proteolytic cleavage site results in the release of free protease; and free protease can cleave another molecule of a protease-inhibitor complex at a proteolytic cleavage site. The complex can also include a binding element conjugated to the protease. Alternatively or additionally, the complex can include a substrate for the protease, where the substrate generates a detectable signal upon cleavage by the protease.

### EXAMPLES

The invention will be further explained by the following Examples, which are intended to be purely exemplary of the invention, and should not be considered as limiting the invention in any way.

### Example 1

### Co-Evolution of a Subtilisin Protease and Substrate

Among enzymes, proteases are unusual in that the substrate is itself a protein. Consequently, optimization of the co-factor site ideally involves engineering both protease and substrate amino acids in the vicinity of the proto-site. In an optimized enzyme, co-factor binding is required for transition state stabilization and substrate binding is required for formation of the co-factor site. This linkage creates high substrate specificity.

A method for co-evolving a triggered enzyme and substrate is illustrated with the serine protease subtilisin. The catalytic aspartic acid 32 of subtilisin was mutated to glycine to create a proto-binding site for small anions. Amino acids in the substrate and in subtilisin were then optimized to create an enzyme which is specific for the sequence FRAM-S (SEQ ID NO:2) and which is triggered by the anion nitrite.

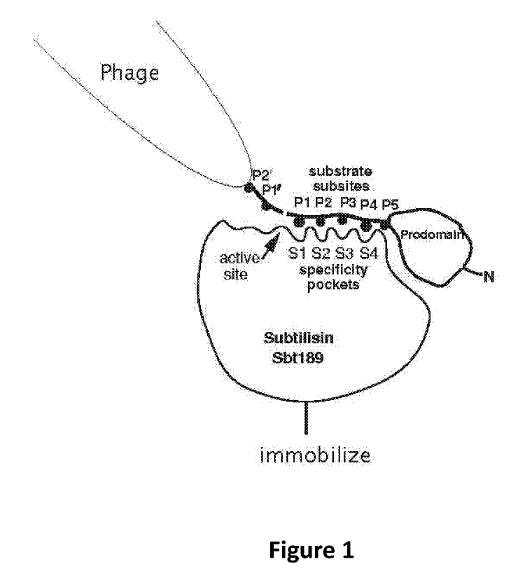

Paradoxically, the method for engineering a high-specificity enzyme begins with damaging the catalytic machinery. It has been shown in the art that in subtilisin, as in all serine proteases, peptide bond cleavage is catalyzed by a nucleophilic serine, which attacks the carbonyl carbon of the scissile peptide bond. The serine is assisted by a general base to increase its nucleophilic character. In most serine proteases, the general base is a histidine coupled to an aspartic acid. In subtilisin, D32 forms a very strong H-bond to NM of H64 which polarizes H64 and allows Nε2 to act as a proton shuttle for the catalytic S221 during acylation and deacylation reactions. In prototype triggered subtilisins previously known in the art, D32 was substituted with alanine, valine, or serine. (Ruan et al., 2004). The D32 mutation creates a protease that is virtually inactive under most conditions. It was shown previously that fluoride, which is a small anion that mimics the function of the catalytic aspartic acid, can rescue some catalytic activity in some D32 mutants of subtilisin. In previous work, these subtilisin mutants were tested for their ability to cut between the methionine and the serine of the amino acid sequence pattern VFKAM-SG (SEQ ID NO:3) in response to triggering by fluoride. The activity of these mutants against this sequence is relatively low, however. For example, the D32A mutant cuts after VFKAM (SEQ ID NO:4) with a rate of 0.6 min⁻¹ in 100 mM fluoride. The sequence VFKAM-SG (SEQ ID NO:3) was carefully designed by the best principles known in the art to optimize interactions between individual substrate amino acids and enzyme sub-sites in the subtilisin. There is a critical deficiency in this approach, however: differences in the binding modes for substrates, transition states and products are subtle and difficult to manipulate via straightforward protein engineering (Hedstrom, 2002; Ruan et al., 2008). These enzymes are slow because neither the cognate sequence nor the triggered enzyme is optimized for each other. The present Example extends and alters the work previously done and shows that it is possible to create very active enzyme-substrate-anion combinations. This can be done using a very powerful method of directed evolution denoted “catch and release” phage display, which is described in detail below and depicted generally in FIG. 1. In essence, the presently disclosed invention recognizes a deficiency in prior art attempts to engineer triggered enzymes by recognizing that, by mutating an enzyme to diminish or abolish activity, the specificity of the enzyme for the original substrate is also altered, typically reduced or abolished. To overcome this deficiency, the present invention uses a selection method that identifies the best substrate for the mutated enzyme by way of a co-evolution or co-selection process. This co-evolution scheme allows for engineering and selection of mutants having altered activities around co-factor triggering, which enables one to engineer/evolve a rudimentary co-factor binding site into a refined co-factor binding site, how to engineer/evolve enzymatic activation with new triggering co-factors, and how to use co-factor triggering to evolve altered specificity.

In this example, an optimal cognate sequence for a D32A mutant of subtilisin denoted SBT189 is disclosed. The ability to separate binding and cleavage reactions with a chemical trigger allows the use of phage display to select for a cognate sequence for SBT189 optimized for cleavage in azide. To perform the selection, an engineered prodomain of subtilisin was synthesized as a fusion protein with the gene III coat protein of the coli phage fd so it is displayed on the surface of phagemid particles according to known phage display procedures.

In this method the P5 to P2′ residues of the prodomain are randomized and expressed as fusions with the g3p protein of M13. Incorporating the random P1 to P5 residues into the prodomain ensures a high baseline binding affinity. The process essentially uses the globular surface of the prodomain as an exo-recognition signal to amplify the binding signal from the substrate binding pockets. Using the prodomain is not essential for this method but is convenient.

In the “catch” phase of phage selection, M13 phage particles tagged with tight binding prodomain mutants are selectively retained by binding to biotinylated SBT189. The biotinylated SBT189 is in turn bound to streptavidin-coated magnetic beads, which are collected on a magnetic particle concentrator. Because of the amplification of the binding signal by the prodomain, the catch phase is a fairly permissive step in the selection process. Subtilisin phage with ≦10 nM K_{D} will be efficiently retained. In the “release” phase, optimal cognates sequences are eluted by mild azide treatment (e.g., 1 mM azide, 2 minutes), which recapitulates catalytic activity of some of the mutated enzymes, resulting in cleavage of the enzyme from the bound prodomain/biotin. Released phagemid are pooled and amplified in E. coli. This is done for three cycles. The consensus motif identified in this selection was:

(SEQ ID NO: 5)
P5 P4 P3 P2 P1 P1′ P2′
L F R A L S A.

Note that the optimal cognate sequence is not the same as the tightest binding sequence. Tight binding substrate sequences can be identified by performing the catch phase of the selection as described above, but afterwards eluting the bound phage in acid rather than by triggered cleavage. The consensus motif from the selection for binding only was:

(SEQ ID NO: 6)
P5 P4 P3 P2 P1 P1′ P2′
L F Y T L M S.

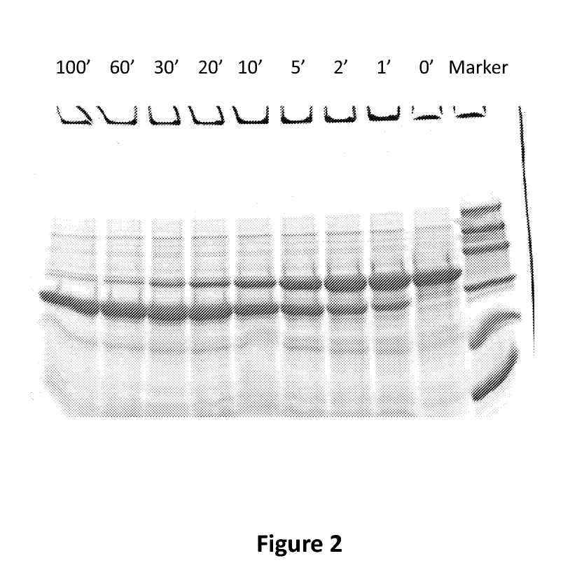

In order to evaluate sequence specificity of SBT189 for the cognate sequence identified by phage display, a gene was constructed to direct the synthesis of a fusion of the 56 amino acid B domain (G_{B}) of streptococcal Protein G to a linker comprising the cognate sequence LFRAL-SA (SEQ ID NO:5) followed by GFP. Accordingly, the protein is denoted “G_{B}-LFRAL-SA-GFP”. The ability of SBT189 to specifically digest the fusion protein in an E. coli extract is shown in FIG. 2.

More specifically, FIG. 2 shows a protein gel of digestion products. Timepoints were collected as indicated. The fusion protein (20 μM) was mixed with 50 nM of SBT189 in 10 mM azide, 0.1M KPi, pH 7.2, at 22° C. The fusion protein was correctly and specifically processed to release “G_{B}-LFRAL” and “SA-GFP”, as confirmed by MALDI-MS.

Seventeen additional G_{B}-GFP fusion proteins were made to test the effect of small variations in the cognate sequence on the rate of the reaction. To obtain detailed mechanistic information about specificity, kinetic analysis was performed using a SBT189-Dabcyl conjugate produced by introducing a free cysteine on the N-terminus of RSUB1 and reacting with Dabcyl-maleimide.

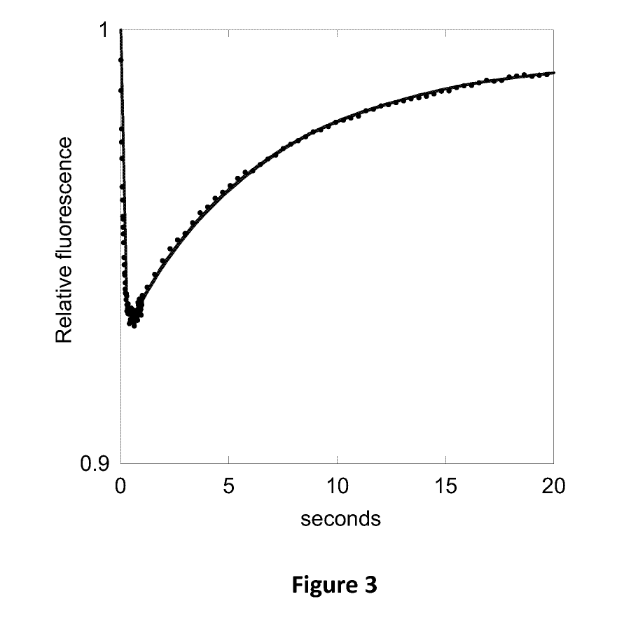

As shown in FIG. 3, the RSUB(Dabcyl) allows quantitation of the formation and decay of the enzyme-substrate complex. When a G_{B}-GFP substrate binds to SBT189 (Dabcyl), GFP fluorescence is quenched by the proximal Dabcyl group. When GFP is cleaved from the complex, GFP fluorescence increases. The plot in FIG. 3 shows the kinetics of the reaction of 0.5 μM “G_{B}-LFRAL-S-GFP” with 3 μM RSUB1(Dabcyl) in 5 mM azide. Fluorescence at 535 nm (excitation=485 nm) decreases as the enzyme-substrate complex forms and increases as it is cleaved to release free GFP.

The fast phase of the reaction measures binding of substrate to the enzyme and the slow phase measures the release of cleaved GFP. By carrying out single turn-over experiments for all substrate variations as a function of enzyme concentration, the values for substrate affinity and acylation rate are compiled for each. Results are summarized below for fusion proteins with detectable cleavage rates.

TABLE 1
Enzymatic Properties of Various Cognate Sequences

| | | acylation | |
|---|---|---|---|
| | K_{S} | rate (k₂) | Relative |
| Cognate variation | (μM) | (s⁻¹) | k₂/K_{S} |
| LFRAL-SA (SEQ ID NO: 5) | 16 | 1.4 | 1.0 |
| LFRAM-SA (SEQ ID NO: 7) | 36 | 1.0 | 0.32 |
| LFQSL-SA (SEQ ID NO: 8) | 66 | 0.23 | 0.04 |
| LYRAL-SA (SEQ ID NO: 9) | 88 | 0.23 | 0.03 |
| LFRAL-MA (SEQ ID NO: 10) | 24 | 0.06 | 0.027 |
| LLRAL-SA (SEQ ID NO: 11) | 670 | 0.59 | 0.01 |
| VFKAM-SG (SEQ ID NO: 3) | 43 | 0.017 | 0.004 |
| LFRAY-SA (SEQ ID NO: 12) | 1900 | 0.17 | 0.001 |

Measurements were made in 0.1M KPO₄, pH 7.2, 5 mM azide, 22° C. Note that the activity of SBT189 versus the cognate sequence optimized by selection (LFRAL-SA; (SEQ ID NO:5)) is 250-times greater than versus the designed cognate (VFKAM-SG; SEQ ID NO:3).

The mutant was further analyzed for its structure. The crystal structure of an inactive form of a triggered subtilisin (catalytic Ser 221 replaced with alanine) in complex with azide and with a substrate that spans the active site was determined at 1.8 Å resolution. FIG. 4 shows the azide anion, the H is 64 side chain, and the scissile region of the substrate. The anion site is buried under the substrate, adjacent to the mutated Ala 32, 8 Å from the scissile peptide. The scissile bond is 2.5 Å from the position where the catalytic nucleophile Ser 221 OG would be (were it not for the S221A mutation). Both P1′ Ser 78′ and P2′ Ala 79′ are in beta conformation, and Ala 79′ forms 2 H-bonds with Ser 218 of the enzyme, in a standard antiparallel beta-sheet interaction. The structure helps explain why anion binding is relatively weak (50 mM, see below). The “catalytic triad” (Ala 221, H is 64, and Ala 32), the oxyanion ligand Asn 155, and the azide anion are indicated. The catalytic nucleophile 221 OG has been modeled, based on the wild-type structure. White lines represent selected interactions under 3.3 A.

### Example 2

### Catch and Release Phage Display Technique

In Example 1, a randomized substrate was presented on the surface of phage particles in order to find an optimized cognate sequence for a specific triggered enzyme. An even more powerful application of catch and release phage display is to present a mutant enzyme library on phage particles in order to evolve the enzyme around a substrate and a trigger.

In phage display of subtilisin, the substrate is a fusion protein comprising an albumin-binding domain (G_{A}), an engineered subtilisin prodomain containing the cognate sequence (P_{COGNATE}), and an IgG binding domain (G_{B}). The prodomain component of this substrate can be thought of as an exo-recognition signal that amplifies binding. The substrate binds via both sub-site interactions and the exo-recognition surface, and has a substrate dissociation constant (K_{S}) of <1 nM. In this scheme the subtilisin is synthesized as a fusion protein on the surface of M13 phage. A random library of subtilisin phage is mixed with the G_{A}-P_{COGNATE}-G_{B} substrate. Phage displaying a misfolded subtilisin or one that has sub-sites that bind poorly to the target sequence are rejected on the basis of non-binding. Phage that bind to substrate are in turn bound to IgG Sepharose via the G_{B} domain in the catch step. Because of the amplification of the binding signal by the prodomain, the catch phase is a fairly permissive step in the selection process. Subtilisin phage with 10 nM K_{B} are efficiently retained. Subtilisin phage that cleave the substrate without the trigger are not retained in the catch step of the selection. This is important for evolving tight regulation as well as specificity.

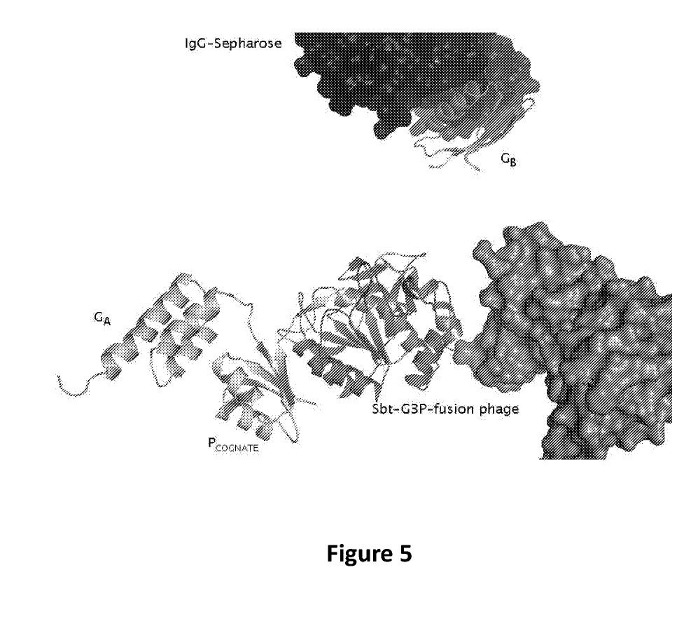

Phage are released by sub-saturating anion concentration. This process is depicted generally in FIG. 5. The released phage in complex with G_{A}-P_{COGNATE}- are then collected on HSA Sepharose. The rate of release of a particular subtilisin-phage reflects both its affinity for anion and the ability of the anion to stabilize the transition state for acylation. Even though substrate binding is amplified by the prodomain, productive substrate interactions in the ternary complex are reflected in anion binding due to their thermodynamic linkage. Thus one can select the two major energetic components contributing to specificity using this system.

Examples 3-4 below discuss useful variations of phage-displayed subtilisin selection methods based on the general concepts provided in Examples 1 and 2.

### Example 3

### Refining the S1 Binding Pocket of a Triggered Subtilisin

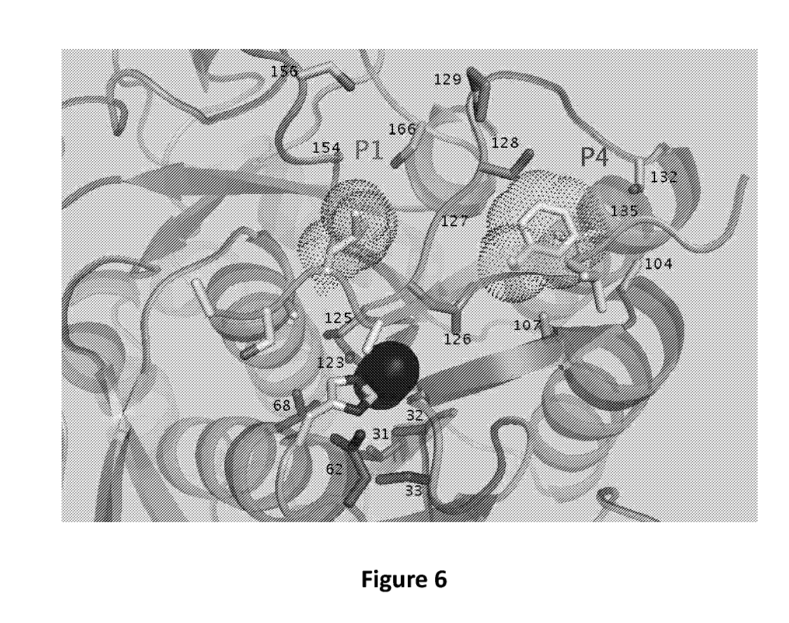

Most subtilisin contacts are made with the first four substrate residues on the acyl side of the scissile bond. The side chain components of substrate binding to subtilisin result primarily from the P1 and P4 amino acids (see, for example, FIG. 4). To test the method of subtilisin display, a subtilisin phage library was constructed with random mutations at position 166 (see also FIG. 6).

The fd gene III fusion phagemid pHEN1 was used to produce fusion phage particles displaying the SBT-g3p fusion proteins on their surfaces. A control selection was performed in which 1.8×10¹¹ SBT -g3p phage particles and 1.5×10¹¹ helper phage were added to 10 pmoles of G_{A}-P_{FRAL-S}-G_{B}. The input of phage corresponds to around 0.2 pmoles of fusion protein. One round of catch and release selection using 20 mM azide resulted in a 350 fold enrichment of phagemid relative to helper the phage.

Mutagenesis of the 166 library was carried out with a single-stranded uracil-containing DNA template according to standard procedures for dut⁻, ung⁻ mutagenesis. The random library was constructed using a degenerate oligonucleotide to randomize codon 166. Transformation of the doubled stranded DNA after the mutagenesis step yielded 10⁹ colony forming units from 1 μg DNA. Sequencing revealed a relatively random distribution of sequences at the target site.

Mutants were selected that cleave the substrate G_{A}-P_{FRAL-S}-G_{B} in response to azide. Phage were bound to the G_{A}-P_{FRAL-S}-G_{B} substrate and collected on IgG-Sepharose. The ability to hydrolyze the fusion protein is selected by washing the beads in 1 mM azide for 5 minutes in the release step. Phage are therefore released or retained from the resin based on the kinetics with which they cleave G_{A}-P_{FRAL} from G_{B} under the triggering condition. Released phage were collected on HSA-Sepharose, acid eluted, neutralized, used to infect fresh E. coli cells, plated out, and colonies counted. Three cycles of selection were carried out. After three rounds of selection, the consensus amino acid at position 166 was threonine. The kinetic properties of the T166 mutant were compared to parent enzyme (SBT189), which has a serine at 166. The T166 mutant hydrolyzed G_{A}-P_{FRAL-S}-G_{B} 1.5-times faster than SBT 189 in 1 mM azide. More significantly, the cleavage rate of T166 in the absence of azide was 3.3-times slower than for SBT189 (0.035 min⁻¹ vs. 0.12 min⁻¹). Thus the ratio of triggered rate to intrinsic rate was increased 5-fold by optimizing a single amino acid position in the S1 subsite. This ratio is a quantitative measure of how tightly the enzyme is regulated by the trigger.

### Example 4

### Evolving Proteases Tightly Regulated with a Different Anion Trigger

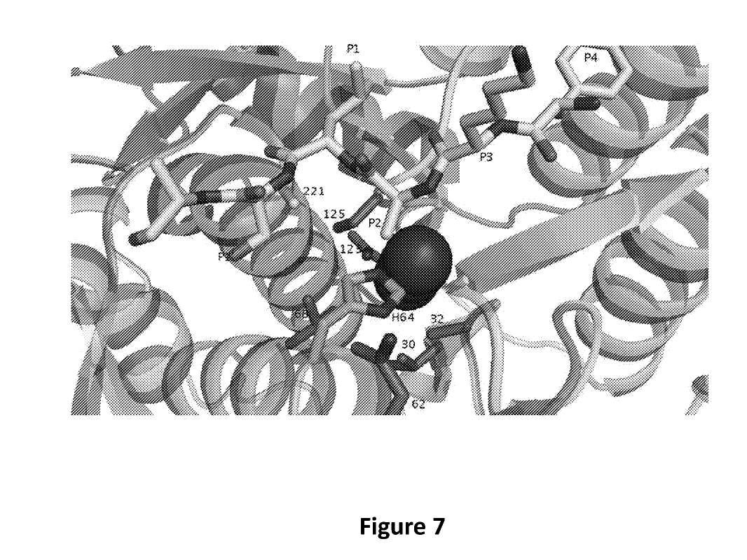

The theory of random library design is that a proper constellation of neighboring residues can create selective binding pockets for substrate amino acids and specific anions. The amino acids chosen for randomization in the anion site library were 30, 32, 33, 62, 68, 123, and 125 (see FIG. 7). The large sequence space generated by mutations at seven positions (1.28×10⁹ variants) produces a high probability of enzymes with the desired triggering properties. Typical libraries represent >10⁹ independent clones. Because the best enzymes are presumably rare, a thorough exploration of the sequence space is desirable and requires powerful selection methods.

Mutants that cleave the substrate G_{A}-P_{FRAL-S}-G_{B} in response to nitrite were selected using the catch and release phage display system of the invention. Phage were bound to the G_{A}-P_{FRAL-S}-G_{B} substrate and collected on IgG-Sepharose. The ability to hydrolyze the fusion protein was selected by washing the beads in 1 mM nitrite for 5 minutes in the release step. Phage are therefore released or retained from the resin based on the kinetics with which they cleave G_{A}-P_{FRAL} from G_{B} under the triggering condition. Released phage were collected on HSA-Sepharose, acid eluted, neutralized, used to infect fresh E. coli cells, plated out and colonies counted. Three cycles of selection were carried out.

After 3 rounds of catch and release selection, the enrichment of colony forming units relative to the input phage increased by around 1000 times. Twenty-four clones from each round of selection were sequenced. Eleven different amino acid sequences were observed in 24 sequences from the third round. Most positions showed strong conservation. Only position 68 tolerated significant variation (7 different amino acids found in 24 clones). The eleven protease mutants from the third round were sub-cloned and expressed in E. coli. Three of these mutants completely cleave G_{A}-P_{FRAL-S}-G_{B} in 5 minutes at 0.1 mM nitrite. These sequences are as follows:

| 30 | 32 | 33 | 62 | 68 | 123 | 125 | |
|---|---|---|---|---|---|---|---|
| I | G | T | S | I | N | P | |
| I | G | T | N | I | N | P | |
| I | G | T | A | I | N | P | |
| V | A | S | N | V | N | S | (parent) |

### Example 5

### Evolving New Specificities by Performing Sequential Selections

Substrate binding pockets and the co-factor site from an interconnected network of binding sites such that binding at one site influences interactions at the others (see FIG. 6). Furthermore, the side chains of an optimal substrate-enzyme combination control the position of the backbone through their interactions with the enzyme binding pockets to achieve an optimum balance between substrate binding and transition state stabilization. Consequently, one can methodically shift specificity and triggering properties of an enzyme in an iterative process. This process is illustrated by a selection of random mutants in the S4 subsite of the subtilisin mutant denoted pT1001. The mutations in pT1001 were identified in the selections described in Examples 3 and 4.

| 30 | 32 | 33 | 62 | 68 | 125 | 166 | |
|---|---|---|---|---|---|---|---|
| I | G | T | S | I | P | T | (pT1001) |
| V | A | S | N | V | S | S | (SBT189) |

A random P4 library was constructed using mutant pT1001 as the subtilisin gene in the parent phagemid. The P4 library comprises random amino acids at positions 104, 107, 128, 130, 132 and 135 (see FIG. 6). The phage library was selected using a substrate sequence (e.g., G_{A}-P_{XRAL-S}-G_{B}), where X=G. Nitrite was used as the triggering anion. The statistics for the three rounds of selection results are as follows:

| | Input phage | Output from IgG | Output from HSA |
|---|---|---|---|
| 1^{st} round | 1.0 × 10¹² cfu | 1.6 × 10⁸ cfu | 3.8 × 10⁵ cfu |
| 2^{nd} round | 2 × 10¹¹ cfu | 3.9 × 10⁶ cfu | 2.0 × 10⁴ cfu |
| 3^{rd} round | 2 × 10¹¹ cfu | 3 × 10⁷ cfu | 3 × 10⁷ cfu |

The convergence in the number of phage released from the IgG resin with the number eluted from HSA resin indicated that a high percentage of the selected phage were displaying enzymes that could both bind the G_{A}-P_{GRAL-S}-G_{B} substrate and cleave the substrate after the GRAL (SEQ ID NO:13) sequence upon addition of nitrite. After three rounds of selection, ten of the phagemid were sequenced. The sequences at the sites of mutation are shown below.

| | | | | | | |
|---|---|---|---|---|---|---|
| | 104 | 107 | 128 | 130 | 132 | 135 |
| PARENT | GCT | ATC | TCA | TCT | TCT | TTA |
| (pT1001) | A | I | S | S | S | L |
| pT2012 | A | I | L | Q | V | L |
| | GCG | ATC | TTC | GAG | TCG | GTC |
| pT2013 | A | I | F | E | S | V |
| | GCT | ATC | ATC | AGC | AGC | CTC |
| pT2014 | A | I | I | S | S | L |
| | GCT | ATC | GTG | TCT | TCT | TTA |
| pT2015 | A | I | V | S | S | L |
| | GCT | ATC | GTC | GGC | AGC | CTG |
| pT2016 | A | I | V | G | S | L |
| | GCT | ATC | TTC | GGC | TCT | TTA |
| pT2017 | A | I | F | G | S | L |
| | GCT | ATC | CTC | GGG | CAC | CTG |
| pT2018 | A | I | L | G | H | L |
| | GCT | ATC | ATC | ACG | TCT | TTA |
| pT2019 | A | I | I | T | S | L |
| | GCT | ATC | CTC | GGC | CAG | CTC |
| pT2020 | A | I | L | G | Q | L |
| | GCT | ATC | CTC | GAC | TCC | CTC |
| pT2021 | A | I | L | D | S | L |

The ten variants from the third round were expressed, purified and assayed for activity against G_{A}-P_{LGRAL-S}-G_{B}. All completely cleave the substrate under the selection conditions (1 mM nitrite, 5 minutes, 25° C.). Further all strongly prefer glycine or alanine at the P4 position of the G_{A}-P_{LXGRAL-S}-G_{B} substrate series relative to the other 18 amino acids. The specificity has thus been changed from the parental preference of (F/Y)RAL- (SEQ ID NO:14) to (G/A)RAL- (SEQ ID NO:15) in one selection cycle.

### Example 6

### Evolving Stability and Facile Folding

Thermal stability and folding rate were determined for 17 mutants from the previous selections. All mutants had melting temperatures above 75° C. and refolded rapidly, refolding into the active conformation after denaturation in acid. Conformational stability and facile folding are required for selection in the phage display methods. Thus these methods provide a means to select these properties in addition to triggered catalysis.

### Example 7

### Theory Underlying Technology

The basic mechanistic framework for understanding a triggered protease is shown below where E is enzyme, S is substrate and where the anion trigger is azide (N₃).

| E + S + N₃ | → | ES + N₃ | | | | | | |
|---|---|---|---|---|---|---|---|---|
| | | ← | | | | | | |
| ↓↑ | | ↓↑ | | | | | | |
| E-N₃ + S | → | ES-N₃ | → | EA-N₃ + P₁ | → | E P₂-N₃ | → | E + |
| P₂ + N₃ | | | | | | | | |
| | | | | | | | ← | |
| | 1 | | 2 | | 3 | | 4 | |

| | | | |
|---|---|---|---|
| Ternary complex formation | acylation | deacylation | product release |

The reaction can be divided into four phases, as noted above. The following describes each step in the reaction pathway and the way each step contributes to specificity.

Ternary complex formation: Step 1 describes the binding of substrate and anion to the enzyme. These binding reactions are thermodynamically linked and in rapid equilibrium relative to the first chemical step (acylation). In the absence of substrate, anion binding to the enzyme is weak as H64 swings out of the active site (chi1=−60° rotamer) and is unavailable to H-bond with the anion. Substrate binding forces H64 into the active site where it is buried beneath P1′ and P2 amino acids of the substrate and forms a H-bond to the anion in the ground state. The cost of pushing H64 into the active site is paid with substrate binding energy. Binding of the anion can repay some of this cost for some substrate sequences. The binding affinity of the anion to the ES complex depends in particular on the P1′ and P2 amino acids. Thus because of the linked equilibrium, substrate sequence exerts an effect on anion affinity in the ground state and creates the first layer of sequence discrimination.

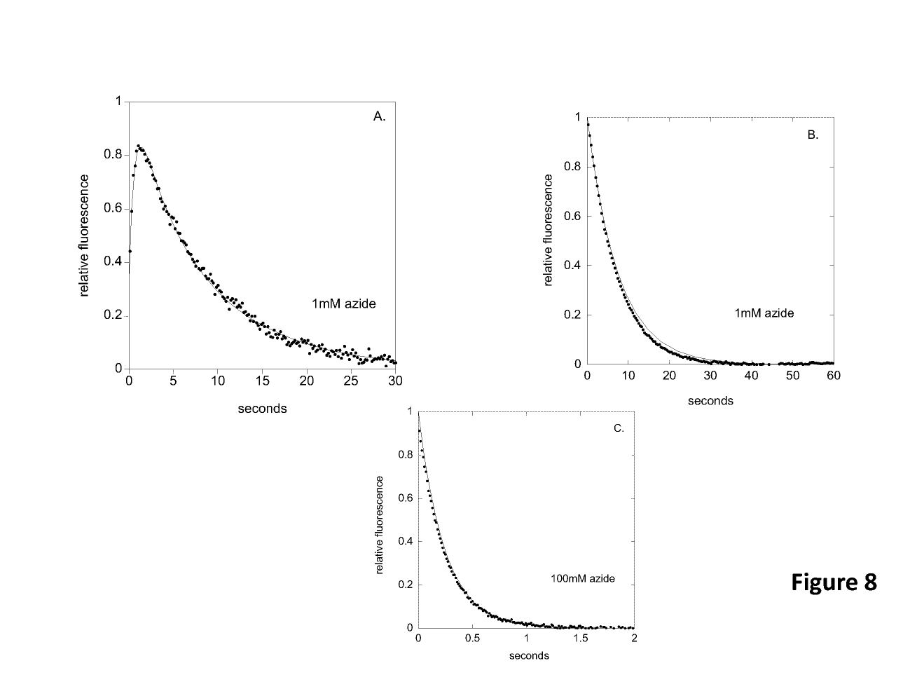

The acylation reaction: Step 2 describes conversion of the ternary complex into an acyl-enzyme with the concomitant release to the C-terminal portion of the substrate. With substrates used in phage display, the G_{B} domain is released concomitantly with the acylation step. If a fluorescent reporter group is attached to subtilisin, a decrease in energy transfer enables time-dependent quantitation of acylation. If anion and Substrate 1 are added simultaneously in a reaction, the kinetics of both formation and decay of the enzyme substrate complex are observed (see FIG. 8A). If the complex is pre-formed with substrate 1 before the introduction of anion, the kinetics reveal a first order conversion of the ternary complex into products (see FIG. 8B-C). In 0.1M KPO₄, pH 7.2 with no azide present, the rate of G_{B} release is 0.0019 s⁻¹ at 22° C. The rate of release in saturating azide is 6.4 s⁻¹, corresponding to an azide dependent rate enhancement of about 3300 fold. The apparent K_{B} for azide is 50 mM. In comparison, the rate of the acylation step for the corresponding wild type active site (with aspartic acid at position 32) is around 20 s⁻¹.

Mechanistically, catch and release phage selection is analogous to the kinetic experiments. The substrate-phage complex is pre-formed in the catch step. Phage are released by sub-saturating with anion (see FIG. 5). The released phage in complex with G_{A}-P_{COGNATE}- are then collected on HSA Sepharose. The rate of release of a particular subtilisin-phage reflects both its affinity for anion and the ability of the anion to stabilize the transition state for acylation. Even though substrate binding is amplified by the prodomain, productive substrate interactions in the ternary complex are reflected in anion binding due to their thermodynamic linkage. Thus one is able to select the two major energetic components contributing to specificity.

The deacylation reaction and product release: A self-quenched FRET peptide that becomes highly fluorescent when cleaved (Dabcyl-EEDKLFRAL-SATE(EDANS)G (SEQ ID NO:16)) was synthesized. This peptide has been used to determine transient state kinetic parameters. Deacylation of SBT189 in 50 mM azide is faster (3.3 s⁻¹) than the wild type counterpart (1.8 s⁻¹). The rate of product (Dabcyl-EEDKLFRAL; (SEQ ID NO:17)) release is 6 s⁻¹ and Kₚ is 1.2 μM. Product binding is not influenced by azide concentration. Strong product binding of subtilisin-phage to G_{A}-P_{COGNATE}- is used for the collection of active mutants in the release step in phage selection (see FIG. 5). Binding is constitutive at this step, however, and is not used to increase selectivity.

Engineered, tightly regulated proteases can be used as the “transistors” of protein based nano-machines. Transistors in electronics are the key element in amplification, detection, and switching of electrical voltages and currents. A protease is a molecular device by which other proteins can be controlled. This concept in employed through biology. Proteases in nature regulate cellular processes from embryogenesis to cell death by linking diverse enzymatic functions together with complex logic gates.

The simplest triggered-protease machines can be used for detection. For example, a nitrite detector consists of an input signal (e.g., an internally quenched FRET peptide used in kinetic analysis) and a nitrite-triggered protease specific for the FRET peptide. Nitrite in the analyte is the regulator and cleaved fluorescent peptide is the output. Due to the rapid breakdown of NO into NO₂, the assay could be used to indicate the NO concentration in body fluids or to assay of nitric oxide synthase activity. Likewise, fluoride detection can be used to detect organofluorophosphate nerve agents (e.g., Sarin and Soman), which spontaneously decompose into fluoride and methylphosphonate. The natural anions formate, acetate, glycolate, lactate, and pyruvate are part of central metabolic pathways and can be used as indicators of metabolic conditions within cells and body fluids. The criteria for a detector protease are low intrinsic cleavage rate, high specificity for the specific anion, and high activity in the presence of that anion. Most sequence specificities would be acceptable provided that they result in tight triggering properties.

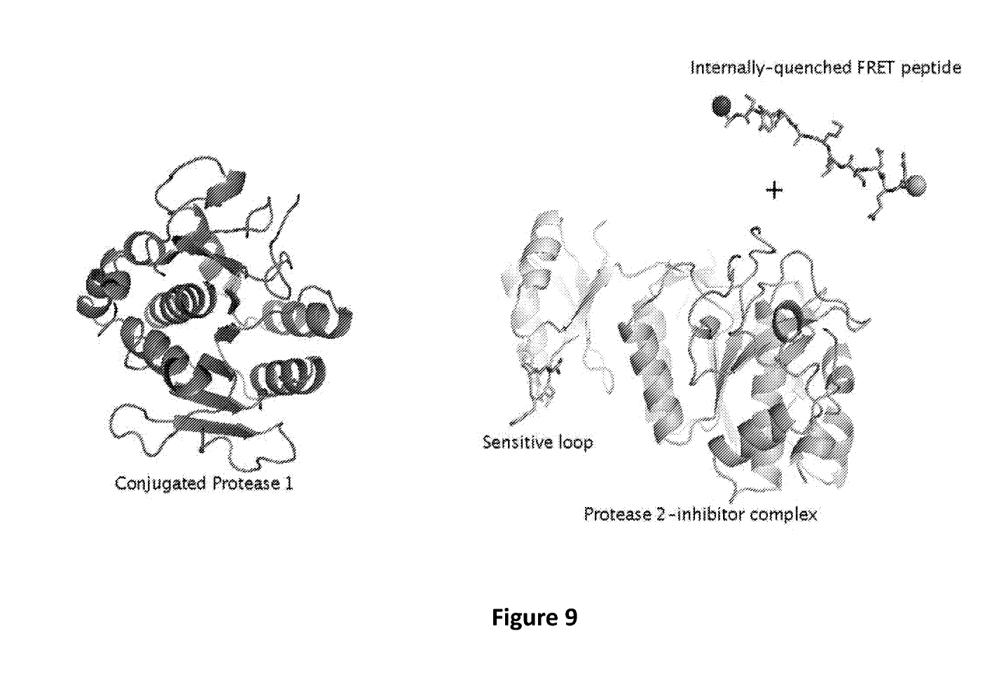

More complex detectors can be built by assembling proteases in series (multiplex detectors). This requires proteases with divergent specificities and different triggers. One protease would activate the next in a cascade of processing events. This is analogous to natural protease cascades such as in blood clotting. An activation cascade can be built on the natural release of subtilisins from their prodomain inhibitors during biosynthesis. Natural prodomains are strong but transient inhibitors due to a protease sensitive site in their globular region. When the sensitive sequence is cleaved, the prodomain unfolds and strong inhibition is lost. This architecture is depicted in the protease activation scheme in FIG. 9 and is discussed in detail in the following Example. Two proteases with different sequence specificities and two triggers create a signal if both triggering anions are present. In terms of logical operators this would be an “AND” gate.

### Example 8

### Use of Engineered Protease in a Signal Amplification Scheme

This Example describes how one or more proteases (such as those evolved in the previous examples) can be used to amplify a binding a signal. A powerful detection system can be built from four basic components: 1) a protease conjugated to a binding molecule, 2) an unconjugated protease, 3) an inhibitor protein which contains a proteolytic cleavage site and 4) a protease substrate which generates a signal upon its cleavage.

A simple version of this system is depicted in FIG. 9. A protease conjugated to the binding molecule is denoted Protease 1, and an unconjugated protease complexed with a cleavable inhibitor is denoted Protease 2. The amplification element of the detector comprises a one to one complex of protease 2 and the inhibitor. The binding between the two is very tight such that the concentration of free protease 2 is extremely low. Addition of a trace amount of Protease 1 to the complex starts a chain reaction in which Protease 1 cleaves the proteolytic cleavage site of the inhibitor, thereby releasing Protease 2. Protease 2 in turn cleaves the proteolytic cleavage site of other inhibitors releasing more Protease 2. Proteases 1 and 2 both cleave the substrate peptide and generate a signal.

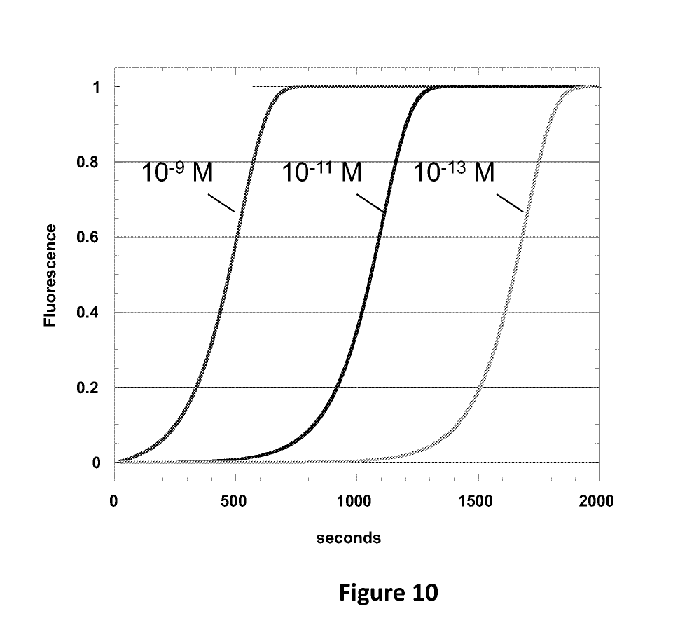

The kinetics of this chain reaction can be seen in FIG. 10. An initial lag phase in the signal from cleaved substrate is observed, followed by rapid increase in signal as the concentration of free Protease 2 increases exponentially during the course of the reaction. The duration of the lag phase is determined by the concentration of Protease 1 used to start the chain reaction. The three critical elements for controlling the protease activation cascade and ultimately determining sensitivity and the signal to noise ratio are very tight inhibition of the protease by the intact inhibitor, rapid cleavage of the inhibitor by free protease, and rapid release of free protease from the cleaved inhibitor. The mechanism of the simple activation cascade is as follows:

IP+P [symbol] PIP

PIP [symbol] P+CP

CP [symbol] C+P

P+S [symbol] PS

PS [symbol] P+Q

In this mechanism, P is free protease, IP is protease inhibitor complex, C is cleaved inhibitor, S is substrate, and Q is cleaved substrate. Note that in this simple mechanism, the conjugated protease and the protease which is initially complexed with the inhibitor can be the same protease and are both simply designated as P in the free state. In the figure the initial concentration of free protease is 10⁻⁹M, 10⁻¹¹ M, and 10⁻¹³ M.

Binding molecules (usually antibodies) are routinely conjugated to enzymes in detection systems to measure the concentration of a specific component in a complex sample. An enzyme-linked immunosorbent assay (ELISA) is the most common example of these detection methods. The present detection methods can use the conjugation of an enzyme to a binding molecule common to ELISA assays, but instead of simply assaying the activity of the conjugated enzyme, the conjugated protease is used to set off the protease cascade. The result is enormous signal amplification. The potential amplification is in some ways analogous to the Polymerase Chain Reaction (PCR) in its ability to use the presence of a few starting molecules to create an exponential increase in signal.

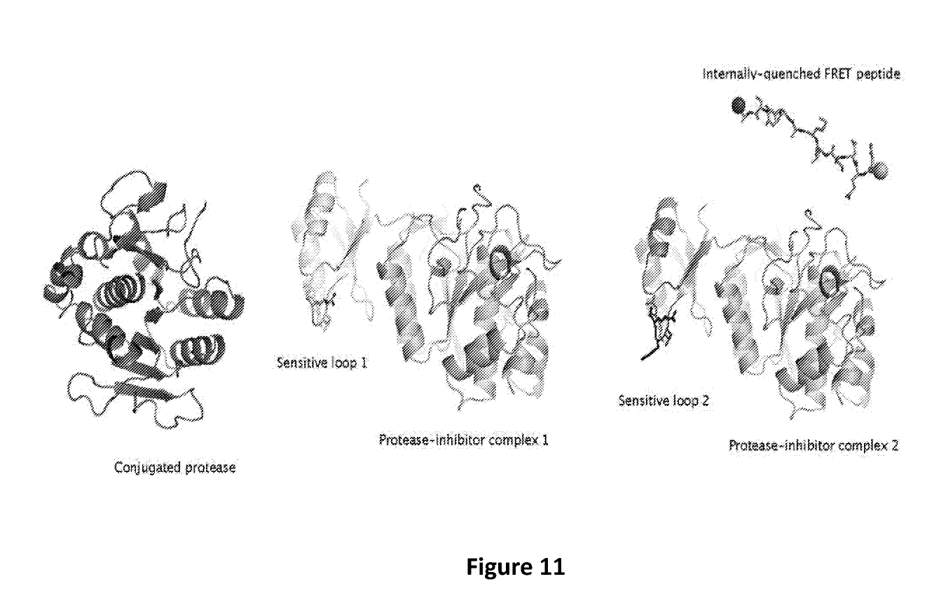

This basic concept enables many variations which potentially improve sensitivity and the signal to noise ratio. The variations also create the potential to simultaneously determine the concentration of multiple components. Two such variations which employ multiple protease inhibitor complexes are shown in FIGS. 11 and 12. In both these examples, a protease in an inhibitor complex is not capable of cleaving its own inhibitor but instead can only release a different protease from its inhibited complex. FIG. 11 shows a reciprocal activation scheme and FIG. 12 shows a serial activation scheme in which the activation signal is transmitted in a linear pathway. Examples of protease-inhibitor combinations which could be used in these schemes would be protease pT1001 in complex with and inhibitor with a GRAL (SEQ ID NO:18) sequence in the sensitive loop, and pT2012 in complex with and inhibitor with an FRAL (SEQ ID NO:19) sequence in the sensitive loop.

Experimental Data: Engineering the subtilisin prodomain as a cleavable inhibitor: Sequencing of the subtilisin gene from Bacillus amyloliquefaciens in the early 1980's revealed that the primary translation product is a pre-pro-protein. A 30 amino acid pre-sequence serves as a signal peptide for protein secretion across the membrane and is hydrolyzed by a signal peptidase. A 77 amino acid sequence, termed a prodomain, was found in between the signal sequence and the 275 amino acid mature subtilisin sequence. The 77 amino acid prodomain is a competitive inhibitor of the active subtilisin (Ki of 5.4×10⁻⁷M) and the entire pro-sequence is required for strong inhibition.

The high resolution structure of a complex between subtilisin and its prodomain is known in the art. The structure shows that the C-terminal portion of the prodomain binds as a substrate into the subtilisin active site and that the globular part of the prodomain has an extensive complementary surface to subtilisin. The isolated prodomain is unfolded but assumes a compact structure with a four-stranded anti parallel β-sheet and two three-turn α-helices in complex with subtilisin. The C-terminal residues extend out from the central part of the pro-domain and bind in a substrate-like manner along subtilisin's active site cleft. Residues Y77, A76, H75, and A74 of the pro-domain become P1 to P4 substrate amino acids, respectively. These residues conform to subtilisin's natural sequence preferences. The folded pro-domain has shape complementary and high affinity to native subtilisin mediated by both the substrate interactions of the C-terminal tail and a hydrophobic interface provided by the β-sheet.

A procedure to select for stable prodomain mutants of subtilisin is known in the art. The selection for stability in that procedure is based on the fact that prodomain binding to subtilisin is thermodynamically linked to prodomain folding. That is, the native tertiary structure of the prodomain is required for maximal binding to subtilisin. If mutations are introduced in regions of the prodomain that do not directly contact subtilisin, their effects on binding to subtilisin are linked to whether or not they stabilize the native conformation. Therefore, mutations that stabilize independent folding of the prodomain increase its binding affinity. Stabilized prodomain variants bind to subtilisin with around 100-times higher affinity than the wild type prodomain.

Characterization of high affinity binding of an engineered prodomain to subtlisin by NMR: Residue-specific exchange rates of 223 amide protons in free and prodomain-complexed subtilisin have been determined in order to understand the energetics of prodomain binding. The engineered version of the subtilisin prodomain used in the studies is denoted proR9 (A23C, K27E, V37L, Q40C, H72K, H75K and T17, M18, S19, T20, M21 replaced with SGIK (SEQ ID NO:20)). ProR9 was engineered to be independently stable. In free subtilisin, amide protons can be categorized according to exchange rate: 74 fast exchangers (rates≧1 hr⁻¹); 52 medium exchangers (rates between 1 hr⁻¹ and 1 days⁻¹); 31 slow exchangers (rates between 1 days⁻¹ and 0.001 days⁻¹). The remaining 66 amide proteins did not exchange detectibly over 9 months (k_{obs}<year⁻¹) and were denoted core protons. Core residues occur throughout the main structural elements of subtilisin. Prodomain binding results in high protection factors (100-1000) in the central β-sheet, particularly in the vicinity of β-strands S5, S6, and S7 and the connecting loops between them.

Characterization of engineered prodomain-protease interactions by x-ray crystallography: It is also known in the art a 1.8 Å resolution structure of a complex between an engineering the prodomain and the azide-triggered protease SBT189. The stabilized version of the prodomain is denoted pG60 and contains the following mutations: replacement of amino acids 17-21 (TMSTM; SEQ ID NO:21) with GFK, and the substitutions A23C, K27E, V37L, Q40C, H72K, A74Y, H75R, and Y77L. pG60 is independently stable and binds to subtilisin with around 100-times higher affinity than the wild type prodomain. As previously observed, the backbone of the substrate inserts between strands 100-104 and 125-129 of subtilisin to become the central strand in an anti-parallel β-sheet arrangement involving seven main chain H-bonds. The wild-type prodomain contains no cysteine, but targeted random mutagenesis with selection led to the introduction of two cysteines that form a well ordered disulfide. This structure is described in detail in the art.

Engineering a protease cleavage site into the prodomain: Using the methods of the present invention, a proteolytic cleavage site for the subtilisin variant SBT189 into a prodomain variant was engineered. In this variant (denoted p5170) the amino acids 18M and 19S were replaced with 18Y and 19K. This creates the amino acid sequence YKTM (SEQ ID NO:22) in a flexible loop between the β1 strand and α-helix 1 of the prodomain. The YKTM (SEQ ID NO:22) sequence can be readily cleaved by free SBT189 in the presence of 10 mM azide. Prodomain variant pS170 also contains the substitution mutations A74F and H73K to improve binding to SBT189 subtilisin in its intact form. A version of the prodomain without the cleavage site for SBT189 was also engineered (denoted pS156). This prodomain contains wild type amino acids at positions 18 and 19 but contains the A74F and H73K substitutions.

Demonstration of a activation cascade: This Example shows how protease activity can be controlled in an activation cascade. To do this complexes of SBT189 were formed with two different prodomain inhibitors. The first complex contained 100 μM of SBT189 and prodomain variant pS156, and the second complex contained 100 μM SBT189 and prodomain variant pS170. To start the activation cascade, 10 nM wild type subtilisin was added to each complex. Wild type subtilisin is able to cleave both the loop sequence MSTM (SEQ ID NO:23) in pS156 and the loop sequence YKTM (SEQ ID NO:27) in pS170. After 5 minutes of digestion, the wild type subtilisin was inactivated by the addition of EDTA to 1 mM and heating to 55° C. for 10 minutes. Azide was then added to the reactions to 10 mM and the activity of free SBT189 subtilisin was then measured as a function of time. The release of free SBT189 from the pS156 complex occurs at a rate of about 1 days⁻¹. This is because the cleavage of the loop sequence MSTM (SEQ ID NO:23) by SBT189 is very slow. In contrast, the complete activation of SBT189 from the pS170 complex occurs within 10 minutes. Because SBT189 can readily cleave the loop sequence YKTL (SEQ ID NO:24), SBT189 is rapidly released after the self-activating chain reaction is initiated by wild type subtilisin. Thus the protease signal from the pS170 complex increases about 10⁴-fold in 10 minutes (from 10 nM to 100 μM).

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1.-28. (canceled)

29. A protease-inhibitor protein complex comprising:
an inhibitor protein comprising a first proteolytic cleavage site, which, when cleaved results in release of the protease from the inhibitor; and
a protease, wherein the protease has proteolytic activity on the first proteolytic cleavage site of the inhibitor.

30. The complex of claim 29, further comprising a binding element conjugated to the protease.

31. The complex of claim 30, wherein the binding element is an antibody.

32. The complex of claim 29, wherein the protease comprises:
a mutation at a residue that is involved in the catalytic activity of the protease, wherein the mutation reduces or abolishes the catalytic activity of the protease for a chosen substrate, and wherein the catalytic activity of the mutant protease can be restored by an exogenous trigger molecule.

33. The complex of claim 29, wherein the protease is a serine protease.

34. The complex of claim 33, wherein the serine protease is subtilisin.

35. A composition of matter comprising:
a protease-inhibitor protein complex comprising:
an inhibitor protein comprising a first proteolytic cleavage site, which, when cleaved results in release of the protease from the inhibitor, and
a protease, wherein the protease has proteolytic activity on the first proteolytic cleavage site of the inhibitor; and
a substrate for the protease, wherein the substrate generates a detectable signal upon cleavage by the protease.

36. The composition of matter of claim 35, further comprising:
a binding element conjugated to the protease.

37. The composition of matter of claim 36, wherein the binding element is an antibody.

38. The composition of matter of claim 35, wherein the protease comprises:
a mutation at a residue that is involved in the catalytic activity of the protease, wherein the mutation reduces or abolishes the catalytic activity of the protease for a chosen substrate, and wherein the catalytic activity of the mutant protease can be restored by an exogenous trigger molecule.

39. The composition of matter of claim 35, wherein the protease is a serine protease.

40. The composition of matter of claim 39, wherein the serine protease is subtilisin.

41. An engineered enzyme that is competent for substrate binding but defective for substrate catalysis in the absence of an exogenous trigger molecule, said enzyme having the following characteristics:
a mutation at a residue that is involved in the catalytic activity of the enzyme, which reduces or abolishes the catalytic activity of the enzyme for a chosen substrate, wherein the catalytic activity of the mutant enzyme can be restored by the exogenous trigger molecule; and another mutation in the mutant enzyme, wherein the other mutation increased the catalytic activity, specificity, or both, of the mutant enzyme for a pre-selected substrate in the presence of the trigger molecule.

42. The engineered enzyme of claim 41, wherein the chosen substrate and the pre-selected substrate are different substrates.

43. The engineered enzyme of claim 41, wherein the engineered enzyme is a protease.

44. The engineered enzyme of claim 43, wherein the engineered enzyme is a serine protease.

45. The engineered enzyme of claim 44, wherein the serine protease is subtilisin.

46. A composition comprising:
the engineered enzyme of claim 41; and
at least one other substance that is compatible with the catalytic activity of the engineered enzyme.

47. The composition of claim 46, wherein the other substance is a trigger molecule that restores the catalytic activity of the engineered enzyme.


SEQUENCE LISTING (24 sequences)

SEQ ID NO: 1 - protein, 6 aa, Artificial Sequence
  Subtilisin related sequence
LFRALS

SEQ ID NO: 2 - protein, 5 aa, Artificial Sequence
  Subtilisin related sequence
FRAMS

SEQ ID NO: 3 - protein, 7 aa, Artificial Sequence
  Subtilisin related sequence
VFKAMSG

SEQ ID NO: 4 - protein, 5 aa, Artificial Sequence
  Subtilisin related sequence
VFKAM

SEQ ID NO: 5 - protein, 7 aa, Artificial Sequence
  Subtilisin related sequence
LFRALSA

SEQ ID NO: 6 - protein, 7 aa, Artificial Sequence
  Subtilisin related sequence
LFYTLMS

SEQ ID NO: 7 - protein, 7 aa, Artificial Sequence
  Subtilisin related sequence
LFRAMSA

SEQ ID NO: 8 - protein, 7 aa, Artificial Sequence
  Subtilisin related sequence
LFQSLSA

SEQ ID NO: 9 - protein, 7 aa, Artificial Sequence
  Subtilisin related sequence
LYRALSA

SEQ ID NO: 10 - protein, 7 aa, Artificial Sequence
  Subtilisin related sequence
LFRALMA

SEQ ID NO: 11 - protein, 7 aa, Artificial Sequence
  Subtilisin related sequence
LLRALSA

SEQ ID NO: 12 - protein, 7 aa, Artificial Sequence
  Subtilisin related sequence
LFRAYSA

SEQ ID NO: 13 - protein, 4 aa, Artificial Sequence
  Subtilisin related sequence
GRAL

SEQ ID NO: 14 - protein, 4 aa, Artificial Sequence
  Subtilisin related sequence
XRAL

SEQ ID NO: 15 - protein, 4 aa, Artificial Sequence
  Subtilisin related sequence
XRAL

SEQ ID NO: 16 - protein, 19 aa, Artificial Sequence
  Subtilisin related sequence
EEDKLFRALSATEEDANSG

SEQ ID NO: 17 - protein, 9 aa, Artificial Sequence
  Subtilisin related sequence
EEDKLFRAL

SEQ ID NO: 18 - protein, 4 aa, Artificial Sequence
  Subtilisin related sequence
GRAL

SEQ ID NO: 19 - protein, 4 aa, Artificial Sequence
  Subtilisin related sequence
FRAL

SEQ ID NO: 20 - protein, 4 aa, Artificial Sequence
  Subtilisin related sequence
SGIK

SEQ ID NO: 21 - protein, 5 aa, Artificial Sequence
  Subtilisin related sequence
TMSTM

SEQ ID NO: 22 - protein, 4 aa, Artificial Sequence
  Subtilisin related sequence
YKTM

SEQ ID NO: 23 - protein, 4 aa, Artificial Sequence
  Subtilisin related sequence
MSTM

SEQ ID NO: 24 - protein, 4 aa, Artificial Sequence
  Subtilisin related sequence
YKTL